(19) United States
(12) Patent Application Publication
(10) Pub. No.: US 2023/0241055 A1
(43) Pub. Date: Aug. 3, 2023

(54) Hydrophilic Degradable Microspheres for Delivering Buprenorphine

(71) Applicants: Occlugel, Les Ulis (FR); Centre National de la Recherche Scientifique (CNRS), Paris (FR); Université Paris-Saclay, Gif Sur Yvette (FR)

(72) Inventors: Anne Beilvert, Massy (FR); Emeline Corus, Janvry (FR); Laurent Bedouet, Les Mans (FR); Laurence Moine, Saint Cloud (FR)

(21) Appl. No.: 18/003,184

(22) PCT Filed: Jul. 7, 2021
(86) PCT No.: PCT/EP2021/025249

§ 371 Date: Dec. 23, 2022

(30) Foreign Application Priority Data
Jul. 7, 2020 (EP) 20305776.5

(51) Int. Cl. A61K 31/485; A61K 9/16

(52) U.S. Cl. CPC A61K 31/485; A61K 9/1635

(57) ABSTRACT

The invention relates to a composition comprising an effective amount of buprenorphine or a pharmaceutically acceptable salt thereof, at least one hydrophilic degradable microsphere comprising a crosslinked matrix, and a pharmaceutically acceptable carrier for administration by injection, the crosslinked matrix being based on at least a) between 10% and 90% of hydrophilic monomer of general formula (I); b) between 0.1 and 30% mol of a cyclic monomer of formula (II); and c) between 5% and 90% of one degradable block copolymer cross-linker, wherein the degradable block copolymer cross linker is linear or star-shaped and presents (CH₂═(CR₁₁))-groups at all its extremities and wherein the degradable block copolymer crosslinker has a partition coefficient P of between −3 and 11.20. The invention also relates to such a composition for use for preventing and/or treating moderate to severe pain, post-surgical pain and/or opioid use disorders.

### FIELD OF THE INVENTION

The present invention relates to hydrophilic degradable microspheres for delivering buprenorphine or a pharmaceutically acceptable salt thereof. In particular, the present invention relates to compositions comprising an effective amount of buprenorphine or a pharmaceutically acceptable salt thereof and hydrophilic degradable microspheres. The present invention also relates to said compositions for use for preventing and/or treating moderate to severe pain, in particular post-surgical pain.

### TECHNICAL BACKGROUND

It is well-known in the art that pain, in particular acute pain, can be treated with opioids such as buprenorphine. Buprenorphine has a 20-40 times higher potency than morphine. The slow dissociation of buprenorphine from the receptor results in a long duration effect. Buprenorphine has been used for years as an analgesic for treatment of moderate to severe pain, in particular in post-surgical patients. Buprenorphine is thus well-known for promoting a strong analgesic action. Buprenorphine has also been used for years for treating opiate addiction.

Oral or sublingual buprenorphine formulations are well-known by the one skilled in the art. These formulations are approved for once or twice administration per day, and may optionally be approved for an administration three times per day. However, the lack of compliance and the possible misuse of these formulations lead to the need of new methods of administering buprenorphine.

In addition, oral formulation of buprenorphine has some disadvantages such as poor gastrointestinal absorption and low systemic availability leading to ineffective pain relief. After oral uptake, buprenorphine undergoes an extensive first-pass through the intestinal wall and hepatic metabolism, resulting in low and variable systemic exposures. Sublingual and transdermal methods of drug administration avoid the first-pass metabolism as they bypass the gastrointestinal tract, thereby improving bioavailability.

The sublingual formulations can improve bioavailability over oral administration through a rapid absorption resulting in high systemic exposure and rapidly attained plasma levels. However, inadvertent swallowing of a part of the sublingual dose can induce a pre-systemic first-pass elimination inducing variation of plasma concentration and large inter-subject variability in drug absorption (Kapil et al; 2013. J Pain Symptom Manage. 46:65-75).

Buprenorphine transdermal patch has been approved for the sustained administration of buprenorphine for the treatment of moderate-to-severe cancers and non-cancer related pain, including post-operative, and musculoskeletal pain such osteoarthritis. Transdermal patches of buprenorphine provide more stable plasma concentrations, controlled and sustained delivery. The seven-day transdermal patches are capable of delivering a constant treatment rate for one week. The delivery rates of transdermal patches are 5-10 or 20 μg/h. These devices yield buprenorphine plasma concentration in a therapeutic threshold with a high level of patient satisfaction. For example, the Butrans® patch at 20 μg/h allows a rapid plasma concentration of 200 pg/mL after four days before a slow decrease up to day 7 until this replacement by another patch (Kapil et al; 2013. J Pain Symptom Manage. 46:65-75). With patches, a stable buprenorphine level can be achieved with the first application and is maintained with repeated weekly applications. However, the use of transdermal patches induces secondary effects, the most common are nausea, constipation, fatigue, dizziness and headache even if the plasmatic concentrations are low (50-70 pg/mL) (Al-Tawil et al; 2013. Eur J Clin Pharmacol. 69:143-149).

Parenteral formulations are also well-known by the one skilled in the art but are poorly tolerated or even prohibited in patients with specific indications and may lead to central side effects. To avoid the secondary effects of systemic applied opioids, their local injection is proposed in order to obtain a peripheral analgesic effect. For example, buprenorphine was injected directly in the operative wounds after surgery in the management of post-surgical pain (Metha et al, 2011, J Anaesthesiol Clin Pharmacol. 27:211-4). Buprenorphine (2 μg/kg) was injected in addition to bupivacaine in the wounds after nephrectomy in 20 patients. The use of rescue analgesics during the first day after surgery was significantly reduced compared to patients treated locally only with bupiviacaine. Direct injection of opioids in the joint cavity after arthroscopy alleviates the post-operative pain (Kalso et al; 2002; Pain. 98; 269-75). For instance, the intra-articular injection of buprenorphine (100 μg) after knee arthroscopy reduced the post-operative pain up to 6 h after surgery. The pain score was lower compared to the control group at 6 h treated with intra-muscular injection of buprenorphine (Varrassi et al; 1999. Acta Anaesthesiol Scand. 43: 51-5).

The rationale behind the local injections of opioids (morphine, buprenorphine) in painful tissues is based on the recent discovery of opioid receptors on the peripheral nerve endings of afferent neurons, the nociceptors. Opioid receptors were first observed on nerve fibres in knee joint of cat (Russell 1987, Neurosci Lett. 23:107-12.) and observations made by Stein et al (1991; N Engl J Med. 325:1123-6) indicate that the opioid receptors in the human knee joint are functional. Intra-articular injection of morphine (1 mg) alleviates pain in patients after arthroscopy surgery more efficiently than intravenous injection of morphine. As a result, peripheral opioid receptors located in the skin, joints and viscera can be targeted for effective analgesia without the central effects seen with systemic opioid treatment. Local delivery of opioids in wounded tissues is a solution to inhibit the pain at its source (Machelska & Celik. 2018. Frontiers in Pharmacology. Vol 9).

The peripheral opioid system allows the development of novel strategies for pain treatment, by divorcing analgesic action from unwanted central side effects. Different solutions for peripheral analgesia are investigated such the development of new opioid drugs with a low diffusion in brain or drug delivery systems (DDS) for local delivery of opioids in peripheral tissues (Machelska & Celik. 2018. Frontiers in Pharmacology. Vol 9). However, a meta-analysis (26 clinical studies, 1531 patients, 13 different surgical interventions) reveals that peripherally applied opioids for acute postoperative pain are ineffective (Nielsen et al; 2015. Acta Anaesthesiol Scand. 59:830-45). The analgesic effect of peripherally applied opioids seems to depend on the presence of preoperative inflammation. The analgesic effect of peripheral opioids has been reported to increase linearly between 6 and 24 h of inflammation in animal studies (Zhou et al; 1998. J Pharmacol Exp Ther. 286:1000-6), indicating that up-regulation of peripheral opioid receptors may be delayed from the induction of inflammation caused by surgery. Significant antalgic effects of locally applied opioids are reported when a pre-existing inflammation was documented before the surgery, for example with patients who underwent hemorrhoidectomy (Tegon et al; 2009, Tech. Coloproctol, 13: 219-24). It is supposed that locally applied opioids are probably removed from the tissue by drainage before the expression of their receptors had occurred. A solution would be a local post-operative injection of injectable DDS that would gradually elute opioids during expression of opioids receptors on sensory neurons in the hours following the surgery.

In the art, several injectable drug delivery systems (DDS) for buprenorphine are reported. Several DDS were designed for the opioid addiction management in patients (review in Rosenthal and Goradia; 2017. Drug Des Devel Ther. 11: 2493-2505). Simon et al; 2006 (Addiction. 101:420-32) developed long-acting biodegradable microspheres composed of poly(L-lactide-co-glycolide) microspheres thanks to sustained release of buprenorphine. After subcutaneous implantation the buprenorphine peaks in plasma after 2-3 days and reach undetectable level after 6 weeks. A rod-shaped implant (26 mm×2.5 mm) buprenorphine (80 mg or 90 mg) (Probuphine®) was made in polymeric matrix composed of ethylene vinyl acetate. After sub-dermal implantation, buprenorphine was delivered at a steady rate for 6 months (White et al; 2009. Drug Alcohol Depend. 2009. 103:37-43). More recently, a novel long-term buprenorphine delivery system to control opioid dependence, maned RBP-6000, was synthesized. Buprenorphine (300 mg) is mixed in PLGA dissolved in N-methyl-pyrrolidone (NMP). After subcutaneous injection, the NMP diffuses out of the polymer and the polymer precipitates, trapping the buprenorphine inside and forming a solid deposit in situ. The deposit releases buprenorphine for one month by diffusion as the polymer degrades (Nasser et al; 2014. Clin Pharmacokinet. 53:813-24). In WO 2017/147285, buprenorphine is encapsulated in poly(D,L-lactide-co-glycolide) microspheres (20-40 μm) for a plasmatic sustained release ranging between weeks and months according to the molecular weight of the degradable poly(D,L-lactide-co-glycolide). A different delivery strategy to deliver buprenorphine is based on the CAM2038 technology, where lipids in contact with aqueous media self-assemble into reversed-phase “water-in-oil” non-lamellar liquid crystal nanoparticle gels (FluidCrystal®; Camurus AB, Lund, Sweden). After injection, through a small 23-gauge needle, the depot in contact with interstitial aqueous fluid transforms into a viscous liquid-crystal gel phase that elutes buprenorphine as the depot degrades. The release is sustained for 1 week up to 1 month (Frost et al; 2019. Addiction. 114: 1416-26). In addition, one composition (Buprenorphine SR™) is described for pain management of small laboratory animals after subcutaneous injection. The delivery system is a biodegradable co-polymer (50:50 molar ratio) of DL-lactide and ε-caprolactone dissolved in a biocompatible organic solvent for a release of buprenorphine for 3 days. Buprenorphine is entrapped after injection upon polymer precipitation, the drug release occurred by diffusion (Foley et al; 2011. J Am Assoc Lab Anim Sci. 50:198-204).

The inventors have noticed the interest represented by buprenorphine delivery systems for the sustained release of buprenorphine. According to the art, it appears a need for novel delivery systems of buprenorphine easily injectable, which degrade quickly and without organic solvent in their composition.

### SUMMARY OF THE INVENTION

In a first aspect, the invention relates to a composition comprising an effective amount of buprenorphine or a pharmaceutically acceptable salt thereof, at least one hydrophilic degradable microsphere comprising a crosslinked matrix, and a pharmaceutically acceptable carrier for administration by injection. The crosslinked matrix is based on at least the following components:
a) from 10 mol % to 90 mol % of a hydrophilic monomer of general formula (I):

(CH₂═CR₁)—CO-D (I)

wherein:
D is O—Z or NH—Z, with Z being —(CR₂R₃)ₘ—CHs, —(CH₂—CH₂—O)ₘ—H, (CH₂—CH₂—O)ₘ—CHs, —(CR₂R₃)ₘ—OH or —(CH₂)ₘ—NR₅R₆ with m being an integer from 1 to 30;
R₁, R₂, R₃, R₄, R₅ and R₆ are, independently of one another, hydrogen atom or a (C₁-C₆)alkyl group;
b) from 0.1 mol % to 30 mol % of a cyclic monomer of formula (II):

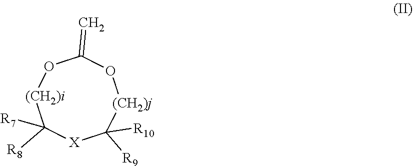

wherein:
R₇, R₈, R₉ and R₁₀ are, independently of one another, a hydrogen atom, a (C₁-C₆)alkyl group or an aryl group;
i and j are independently of one another an integer chosen between 0 and 2; and
X is a single bond or an oxygen atom;
   and
c) from 5 mol % to 90 mol % of a linear or star-shaped degradable block copolymer cross-linker having a partition coefficient P of between −3 and 11.20, or a hydrophobic/hydrophilic balance R between 1 and 20, said degradable block copolymer cross-linker having the formula:

(CH₂═CR₁₁)—CO—Xₙ-PEGₚ-Xₖ—CO—(CR₁₁═CH₂) (IIIa); or

W(PEGₚ-Xₙ—O—CO—(CR₁₁═CH₂))_{z} (IIIc);
wherein
   R₁₁ is independently a hydrogen atom or a (C₁-C₆)alkyl group;
   X is independently PLA, PGA, PLGA, PCL or PLAPCL;
   n and k are independently integers from 1 to 150;
   p is an integer from 1 to 100;
   W is a carbon atom, a C₁-C₆-alkyl group or an ether group comprising 1 to 6 carbon atoms;
   z is an integer from 3 to 8;
      wherein mol % of components a) to c) are expressed relative to the total number of moles of compounds a), b) and c).

In a second aspect, the invention relates to the composition of the invention for use for preventing and/or treating moderate to severe pain; post-surgical pain, advantageously for locally treating post-surgical pain; or opioid use disorders.

In a third aspect, the invention relates to the hydrophilic degradable microsphere of the invention for use for delivering an effective amount of buprenorphine or a pharmaceutically acceptable salt thereof to a subject in need thereof.

In a fourth aspect, the invention relates to a pharmaceutical kit comprising:

i) at least one hydrophilic degradable microsphere of the invention in association with a pharmaceutically acceptable carrier for administration by injection;
ii) an effective amount of buprenorphine or a pharmaceutically acceptable salt thereof; and
iii) optionally an injection device,
the hydrophilic degradable microsphere and the buprenorphine or a pharmaceutically acceptable salt thereof being packed separately.

### FIGURES

FIG. 1: Effect of crosslinker composition at 5 mol % on buprenorphine loading on degradable MS in water for 2 h (target loading of 1 mg/mL). Comparisons were done relative to the crosslinker PEG₁₃-PLA₁₂ using the non-parametric Mann-Whitney (MW) test. Significance was set at p<0.05. Data are means.

FIG. 2: Effect of 3 concentrations (5-15-30 mol %) of 2 crosslinkers (PEG₁₃-PLA₇-PCL₃ and PEG₁₃-PCL₈) on buprenorphine loading on degradable MS in water for 2 h (target loading of 1 mg/mL). For each group of crosslinker, comparisons between the group at 5 mol % and the groups at 15 mol % or 30 mol % were done using the non-parametric Mann-Whitney test. Significance was set at p<0.05 for PEG₁₃-PLA₇-PCL₃ crosslinker (*) and for the PEG₁₃-PCL₈ crosslinker (#). Data are means.

FIG. 3: Effect of the concentration of PEG₁₃-PCL₈ crosslinker (5 mol % or 15 mol %) on loading level of buprenorphine. A: buprenorphine loading (mg/mL); B: efficacy of buprenorphine loading (%). For each level of drug loading, comparisons were done relative to the 5 mol % of crosslinker PEG₁₃-PCL₈ using the non-parametric Mann-Whitney test. Significance was set at p<0.05. Data are means.

FIG. 4: Effect of the linear crosslinkers at 5 mol % on the initial release of buprenorphine from degradable microspheres during swelling in water (A), after 5 h in PBS (B), after 24 h in PBS (C) and after 48 h in PBS (D). The release values are cumulative. The Kruskal-Wallis (KW) non-parametric test was used to compare the effect of crosslinker composition on buprenorphine release at different times. Significance was set at p<0.05. Data are means.

FIG. 5: Effect of crosslinker at 5 mol % on the in vitro release of buprenorphine in PBS. The buprenorphine loading objective was 1 mg/mL. *Complete degradation of MS1, MS2, MS3 and MS14 at day 4, 7, 10 and 1, respectively. MS4, MS5, MS6 and MS15 were not degraded during the in vitro study.

FIG. 6: Effect of 3 concentrations (5-15-30 mol %) of two crosslinkers (PEG₁₃-PLA₇-PCL₃ and PEG₁₃-PCL₈) on the initial release of buprenorphine during sterile MS swelling in water (A), after 5 h in PBS (B), after 24 h in PBS (C) and after 48 h in PBS (D). The release values are cumulative. For each group of crosslinker, comparisons between the group at 5 mol % and the groups at 15 mol % or 30 mol % were done using the non-parametric Mann-Whitney test. Significance was set at p<0.05 for PEG₁₃-PLA₇-PCL₃ crosslinker (*) and for the PEG₁₃-PCL₈ crosslinker (#), respectively. NS: non-significant.

FIG. 7: Effect of crosslinker concentration (5 mol %, 15 mol %, 30 mol %) on the in vitro sustained release of buprenorphine in PBS. A: Concentration effect of the PEG₁₃-PLA₇-PCL₃ crosslinker (target 1 mg/mL). B: Concentration effect of the PEG₁₃-PCL₈ crosslinker (target 1 mg/mL). *complete degradation of MS3 at day 7; the other microspheres were not degraded during the in vitro study.

FIG. 8: Effect of buprenorphine payload on drug release from degradable microspheres containing 5 mol % or 15 mol % of crosslinker PEG₁₃-PCL₈. The release was observed during 16 days. Microspheres were not degraded during the in vitro study.

FIG. 9: Effect of the crosslinker concentration on microsphere degradation time. 250 μL of sterile microspheres (MS5, MS8, MS11 and MS12) were incubated in 10 mL of 100 mM sodium hydroxide at 37° C. (150 rpm). The time (minutes) of MS disappearance was recorded.

FIG. 10: In vitro release in PBS of buprenorphine after the extemporaneous loading on dry MS (50-100 μm). After the fast loading step (5 min) of buprenorphine on dry and sterile microspheres, the drug release follows the degradation of microspheres. Release from MS3 (loading value=0.89 mg/mL) which degraded in 10 days of incubation in PBS. Release during 1 month from MS11 (loading value=2.56 mg/mL) which degraded more slowly in PBS (>6 months). MS16 was degraded in 2 days after a significant burst of buprenorphine (50%) 10 min after addition of PBS.

FIG. 11: Plasmatic concentrations of buprenorphine (BPN) after single subcutaneous injection of degradable microspheres loaded with buprenorphine.

FIG. 12: Tissular concentrations of buprenorphine one month after single subcutaneous injection of degradable microspheres loaded with buprenorphine

FIG. 13: Size distribution of degradable microspheres MS3 and MS4 containing 5 mol % of crosslinker PEG₁₃-PLA₇-PCL₃ and PEG₁₃-PLA₅-PCL₄, respectively.

### DETAILED DESCRIPTION OF THE INVENTION

The inventors have discovered hydrophilic degradable microspheres that may be used as biocompatible drug carrier for local delivery and that present affinity with the active ingredient buprenorphine and salts thereof.

The inventors surprisingly found that degradable hydrophilic microspheres made of a crosslinked hydrogel displays strong affinity for buprenorphine and salts thereof allowing an efficient loading in few minutes and the subsequent sustained release. The present invention offers the possibility after mini-invasively injection, of a local sustained release of low dose of buprenorphine or salts thereof after surgical intervention in the aim to get a pain relief for several days. This approach would allow when the patient returns home to reduce the use of various analgesics, including opiates avoiding the diversion of prescription.

The inventors have thus discovered a buprenorphine or salts thereof delivery system that is free of organic solvent, that presents a tuneable degradation time from day to months, that is easy to load (in water, at room temperature), that allows a sustained and complete drug release and that avoids immediate burst and intense inflammatory reaction.

The buprenorphine or salts thereof delivery system of the invention allows an extended and local release of buprenorphine or salts thereof. For example, the composition of the invention allows to extend post-surgical pain management period by local delivery of low amounts of buprenorphine or salts thereof. Such a delivery avoids central side effects while allowing an extended duration of peripheral analgesia without the need of several administrations of opioids. For example, the composition of the invention may be useful for pain relief for a few days with a single injection.

### Definition

As used herein, the expression “matrix based on” means a matrix comprising a mixture of at least components (a) to (c) and/or a matrix resulting from the reaction, in particular from the polymerization, between at least components (a) to (c). Hence, components (a) to (c) can be seen as the starting components that are used for the polymerization (e.g. heterogenous medium polymerization) of the matrix.

The expression “reaction mixture” as used herein designates the polymerisation medium including any components taking part to the polymerisation. The reaction mixture typically comprises at least components a), b), c) as defined in the claims and in this description, optionally a polymerization initiator such as, for example, t-butyl peroxide, benzoyl peroxide, azobiscyanovaleric acid (also called 4,4′-Azobis(4-cyanopentanoic acid)), AIBN (azobisisobutyronitrile), or 1,1′-azobis(cyclohexane carbonitrile) or optionally one or more photo-initiators such as 2-hydroxy-4′-(2-hydroxyethoxy)-2-methylpropiophenone (106797-53-9); 2-hydroxy-2-methylpropiophenone (Darocur® 1173, 7473-98-5); 2,2-dimethoxy-2-phenylacetophenone (24650-42-8); 2,2-dimethoxy-2-phenyl acetophenone (Irgacure®, 24650-42-8) or 2-methyl-4′-(methylthio)-2-morpholinopropiophenone (Irgacure®, 71868-10-5), and at least one solvent, preferably a solvent mixture comprising an aqueous solvent and an organic solvent such as an apolar aprotic solvent, for example a water/toluene mixture and optionally any suitable components as described herein (e.g. stabilizer such as polyvinyl alcohol).

Thus, in the present description, expressions such as “the [starting component X] is added to the reaction mixture in an amount of between YY % and YYYY %” and “the cross-linked matrix is based on [starting component X] in an amount of between YY % and YYYY %” are interpreted in a similar manner. Similarly, expressions such as “the reaction mixture comprises at least [starting component X]” and “the cross-linked matrix is based on at least [starting component X]” are interpreted in a similar manner.

In the context of the invention, “organic phase” of the reaction mixture means the phase comprising the organic solvent and the compounds soluble in said organic solvent, in particular the monomers, and the polymerization initiator.

As used herein, the terms “(C_{X}-C_{Y})alkyl group” mean a saturated monovalent hydrocarbon chain, linear or branched, containing X to Y carbon atoms, X and Y being integers between 1 and 36, preferably between 1 and 18, in particular between 1 and 6. Examples are methyl, ethyl, propyl, isopropyl, butyl, isobutyl, sec-butyl, tert-butyl, pentyl or hexyl groups.

As used herein, the terms “aryl group” and “(C_{X}-C_{Y})aryl” mean an aromatic hydrocarbon group, preferably having X to Y carbon atoms, X and Y being integers between 6 and 36, preferably between 6 and 18, in particular between 6 and 10. The aryl group may be monocyclic or polycyclic (fused rings). Examples are phenyl or naphthyl groups.

As used herein, the terms “partition coefficient P” mean the ratio of concentrations of a compound in a mixture of two immiscible solvents at equilibrium: water and 1-octanol. This ratio is therefore a comparison of the solubilities of the solute in these two liquids. Hence the octanol/water partition coefficient measures how hydrophilic (octanol/water ratio<1) or hydrophobic (octanol/water>1) a compound is. Partition coefficient P may be determined by measuring the solubilities of the compound in water and in 1-octanol and by calculating the ratio solubility in octanol/solubility in water. Partition P may also be determined in silico using Chemicalize provided by ChemAxon.

As used herein, the hydrophobic/hydrophilic balance R of the degradable crosslinkers is quantified by the ratio of the number of hydrophobic units to the number of hydrophilic units according the following equation:

$$R = \frac{{N}_{hydrophobic units}}{{N}_{hydrophilic units}}$$

with N being an integer and representing the number of unit(s).

For example, for the crosslinkers that can be used in the present invention, R is:

$$R = \frac{{N}_{CHlactide} + {N}_{CH 3 lactide} + {N}_{C H 2 g l y c olide} + 5 \times {N}_{CH 2 caprolactone}}{{N}_{EO unit}}$$

with N being an integer and representing the number of unit(s).

As used herein, the terms “degradable microsphere” mean that the microsphere is degraded or cleaved by hydrolysis in a mixture of degradation products composed of low-molecular-weight compounds and water-soluble polymer chains having molecular weights below the threshold for renal filtration of 50 kg mol⁻¹.

As used herein, the expression “hydrophilic degradable microsphere” means a degradable microsphere containing from 10% to 90% of a hydrophilic monomer which allows a good compatibility with the aqueous media and a low adhesion to solid surface (syringes, needles, catheters).

As used herein, the expression “between X and Y” (wherein X and Y are numerical value) means a range of numerical values in which the limits X and Y are inclusive.

As used herein, the expression “immediate release (IR)” of an active ingredient means the rapid release of the active ingredient from the formulation to the location of delivery as soon as the formulation is administered.

As used herein, the expression “extended-release” of an active ingredient means either the “sustained-release (SR)” or the “controlled-release (CR)” of active ingredients from the formulation to the location of delivery at a predetermined rate for an extended period of time and maintaining a constant active ingredient level for this period of time with minimum side effects. The controlled-release (CR) differs from the sustained-release (SR) in that CR maintains drug release over a sustained period at a constant rate whereas SR maintains drug release over a sustained period but not at a constant rate.

As used herein, the expression “sustained-release” of an active ingredient means an extended-release (as defined above) of an active ingredient from the formulation to the location of delivery in order to maintain for a certain predetermined time the drug in tissue of interest at therapeutic concentrations by means of an initial dose portion.

As used herein, the expression “controlled-release (CR)” of an active ingredient means an extended-release (as defined above) of an active ingredient from the formulation to the location of delivery that provides some control of temporal or spatial nature, or both.

As used herein, the term “pharmaceutically acceptable” is intended to mean what is useful to the preparation of a pharmaceutical composition, and what is generally safe and non toxic, for a pharmaceutical use.

As used herein, the terms «pharmaceutically acceptable salt» mean a salt of a compound which is pharmaceutically acceptable, as defined above, and which possesses the pharmacological activity of the corresponding compound. Such salts comprise:

(1) hydrates and solvates,
(2) acid addition salts formed with inorganic acids such as hydrochloric, hydrobromic, sulfuric, nitric and phosphoric acid and the like; or formed with organic acids such as acetic, benzenesulfonic, fumaric, glucoheptonic, gluconic, glutamic, glycolic, hydroxynaphtoic, 2-hydroxyethanesulfonic, lactic, maleic, malic, mandelic, methanesulfonic, muconic, 2-naphtalenesulfonic, propionic, succinic, dibenzoyl-L-tartaric, tartaric, p-toluenesulfonic, trimethylacetic, and trifluoroacetic acid and the like, and
(3) salts formed when an acid proton present in the compound is either replaced by a metal ion, such as an alkali metal ion, an alkaline-earth metal ion, or an aluminium ion; or coordinated with an organic or inorganic base. Acceptable organic bases comprise diethanolamine, ethanolamine, N-methylglucamine, triethanolamine, tromethamine and the like. Acceptable inorganic bases comprise aluminium hydroxide, calcium hydroxide, potassium hydroxide, sodium carbonate and sodium hydroxide.

Molar percentage is abbreviated herein as mol %.

### Microsphere

According to the present invention, the hydrophilic degradable microsphere comprises a crosslinked matrix that is based on, preferably that results from the polymerization of, at least the following components:
a) from 10 to 90 mol % of a hydrophilic monomer of general formula (I):

(CH₂═CR₁)—CO-D (I)

wherein:
D is O—Z or NH—Z, with Z being —(CR₂R₃)ₘ—CHs, —(CH₂—CH₂—O)ₘ—H, —(CH₂—CH₂—O)ₘ—CHs, —(CR₂R₃)ₘ—OH or —(CH₂)ₘ—NR₅R₆ with m being an integer from 1 to 30;
R₁, R₂, R₃, R₄, R₅ and R₆ are, independently of one another, hydrogen atom or a (C₁-C₆)alkyl group;
b) from 0.1 to 30 mol % of a cyclic monomer of formula (II):

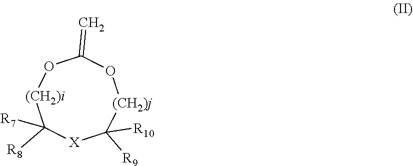

wherein:
R₇, R₈, R₉ and R₁₀ are, independently of one another, a hydrogen atom, a (C₁-C₆)alkyl group or an aryl group;
i and j are independently of one another an integer chosen between 0 and 2; and
X is a single bond or an oxygen atom;
   and
c) from 5 to 90 mol % of a degradable block copolymer cross-linker, wherein the degradable block copolymer crosslinker is linear or star-shaped and presents (CH₂═(CR₁₁))-groups at all its extremities, each R₁₁ being independently of one another hydrogen atom or a (C₁-C₆)alkyl group, and wherein the degradable block copolymer crosslinker has a partition coefficient P between −3 and 11.2, typically between 0.5 and 11.2 or a hydrophobic/hydrophilic balance R between 1 and 20;
   wherein mol % of components a) to c) are expressed relative to the total number of moles of compounds a), b) and c).

The partition coefficient P is determined in silico using Chemicalize provided by ChemAxon.

When the hydrophilic degradable microsphere comprises a crosslinked matrix that is based on further monomers (see monomer e) below), the mol % of components a) to c) are expressed relative to the total number of moles of compounds a), b), c) and e).

The terms “hydrophilic monomer” mean a monomer having a high affinity for water, i.e. tending to dissolve in water, to mix with water, to be wetted by water, or that gives rises to a polymer capable of swelling in water after polymerization.

The block copolymer cross-linker is a degradable block copolymer cross-linker, i.e. a polymer with linear (or radial) arrangement of different blocks joined by covalent bond. In a degradable block copolymer the covalent bond are degradable such as ester bonds, amide bonds, anhydride bond, urea bond or polysaccharidic bond and here specifically ester bonds.

When X is a single bond, it is meant that the carbon atoms bearing R₇, R₈, R₉ and R₁₀ groups are directly linked via a single bond.

The hydrophilic degradable microsphere is a swellable degradable (i.e. hydrolyzable) cross-linked polymer in the form of spherical particle having a diameter after swelling in physiological saline solution (i.e. normal saline solution) ranging between 20 μm and 1200 μm. In particular the cross-linked polymer is constituted of at least one chain of polymerized components a), b) and c) as defined above.

In the context of the invention, a polymer is swellable if it has the capacity to absorb liquids, in particular water. The expression “size after swelling” means thus that the size of the microspheres is considered after the polymerization and sterilization steps that take place during their preparation.

Advantageously, the microsphere of the invention has a diameter after swelling in physiological saline solution (i.e. normal saline solution) of between 20 μm and 100 μm, 40 μm and 150 μm, 100 μm and 300 μm, 300 μm and 500 μm, 500 μm and 700 μm, 700 μm and 900 μm or 900 μm and 1200 μm, as determined by optical microscopy. Microspheres are advantageously small enough in diameter to be injected through needles, catheters or microcatheters with internal diameters ranging from a few hundred micrometres to more than one millimetre.

The hydrophilic monomer a) is of general formula (I):

(CH₂═CR₁)—CO-D (I)

wherein:
D is O—Z or NH—Z, with Z being —(CR₂R₃)ₘ—CH₃, —(CH₂—CH₂—O)ₘ—H, —(CH₂—CH₂—O)ₘ—CH₃, —(CR₂R₃)ₘ—OH or —(CH₂)ₘ—NR₅R₆ with m being an integer from 1 to 30;
R₁, R₂, R₃, R₄, R₅ and R₆ are, independently of one another, hydrogen atom or a (C₁-C₆)alkyl group.

Advantageously, the hydrophilic monomer a) is selected from the group consisting of sec-butyl acrylate, n-butyl acrylate, t-butyl acrylate, t-butyl methacrylate, methylmethacrylate, N-dimethyl-aminoethyl(methyl)acrylate, N, N-dimethylaminopropyl-(meth)acrylate, t-butylaminoethyl (methyl)acrylate, N,N-diethylaminoacrylate, acrylate terminated poly(ethylene oxide), methacrylate terminated poly(ethylene oxide), methoxy poly(ethylene oxide) methacrylate, butoxy poly(ethylene oxide) methacrylate, acrylate terminated poly(ethylene glycol), methacrylate terminated poly(ethylene glycol), methoxy poly(ethylene glycol) methacrylate, butoxy poly(ethylene glycol) methacrylate; advantageously acrylate terminated poly(ethylene glycol), methacrylate terminated poly(ethylene glycol), methoxy poly(ethylene glycol) methacrylate, butoxy poly(ethylene glycol) methacrylate.

In some embodiments, in the formula (I), when Z is —(CR₂R₃)ₘ—CH₃ or —(CR₂R₃)ₘ—OH, m is preferably an integer from 1 to 6.

In some embodiments, in the formula (I), when Z is —(CR₂R₃)ₘ—CHs, Z is preferably a C₁-C₆-alkyl group.

In some embodiments, in the formula (I), when Z is —(CR₂R₃)ₘ—OH, R₂ and R₃ are preferably hydrogen and m is an integer from 1 to 6.

In some embodiments, the hydrophilic monomer a) is of general formula (I):

(CH₂═CR₁)—CO-D (I)

wherein:
D is O—Z, with Z being —(CH₂—CH₂—O)ₘ—H or —(CH₂—CH₂—O)ₘ—CHs, with m being an integer from 1 to 30;
R₁ is hydrogen atom or a (C₁-C₆)alkyl group, preferably a methyl.

More advantageously, the hydrophilic monomer a) is poly(ethylene glycol) methyl ether methacrylate (m-PEGMA).

The amount of hydrophilic monomer a) typically ranges from 10 mol % to 90 mol %, preferably from 30 mol % to 85 mol %, more preferably from 30 mol % to 80 mol %, relative to the total number of moles of components a), b) and c) (or relative to the total number of moles of components a), b), c) and e) when e) is present—see below).

The hydrophilic monomer a) is advantageously present in the reaction mixture in an amount ranging from 10 mol % to 90 mol %, preferably from 30 mol % to 85 mol %, more preferably from 30 mol % to 80 mol %, relative to the total number of moles of components a), b) and c) (or relative to the total number of moles of components a), b), c) and e) when e) is present—see below).

Component b) is a cyclic monomer of formula (II) as defined above, wherein:
R₇, R₈, R₉ and R₁₀ are, independently of one another, hydrogen atom, a (C₁-C₆)alkyl group or an aryl group;
i and j are independently of one another an integer chosen between 0 and 2;
X is a single bond or an oxygen atom.

Advantageously, component b) is a cyclic monomer of formula (II) as defined above, wherein:
R₇, R₈, R₉ and R₁₀ are, independently of one another, hydrogen atom or a (C₅-C₇)aryl group;
i and j are independently of one another an integer chosen between 0 and 2;
X is a single bond or an oxygen atom.

Advantageously, component b) is a cyclic monomer of formula (II) as defined above, wherein:
R₇, R₈, R₉ and R₁₀ are, independently of one another, hydrogen atom or a (C₅-C₇)aryl group;
i and j are independently of one another an integer chosen between 0 and 1;
X is a single bond or an oxygen atom.

Advantageously, the component b) is selected from the group consisting of 2-methylene-1,3-dioxolane, 2-methylene-1,3-dioxane, 2-methylene-1,3-dioxepane, 2-methylene-1,3,6-trioxocane and derivatives thereof, in particular benzo derivatives and phenyl substituted derivatives, advantageously from the group consisting of 2-methylene-1,3-dioxolane, 2-methylene-1,3-dioxane, 2-methylene-1,3-dioxepane, 2-methylene-4-phenyl-1,3-dioxolane, 2-methylene-1,3,6-trioxocane and 5,6-benzo-2-methylene-1,3dioxepane, more advantageously from the group consisting of 2-methylene-1,3-dioxepane, 5,6-benzo-2-methylene-1,3dioxepane and 2-methylene-1,3,6-trioxocane. More advantageously, the component b) is 2-methylene-1,3-dioxepane or 2-methylene-1,3,6-trioxocane.

The amount of component b) typically ranges from 0.1 mol % to 30 mol %, preferably from 1 mol % to 20 mol %, and in particular from 1 mol % to 10 mol %, relative to the total number of moles of components a), b) and c) (or relative to the total number of moles of components a), b), c) and e) when e) is present—see below). In some embodiments, the amount of component b) is about 10 mol %.

The cyclic monomer b) of general formula (II) is advantageously present in the reaction mixture in an amount ranging from 0.1 mol % to 30 mol %, preferably from 1 mol % to 20 mol %, and in particular from 5 mol % to 15 mol % or from 1 mol % to 10 mol %, relative to the total number of moles of components a), b) and c). In some embodiments, the amount of component b) is about 10 mol %.

Component c) is a degradable block copolymer crosslinker, wherein the degradable block copolymer crosslinker is linear or star-shaped and presents (CH₂═(CR₁₁))-groups at all its extremities, each R₁₁ being independently of one another hydrogen atom or a (C₁-C₆)alkyl group.

The degradable block copolymer crosslinker has a partition coefficient P of between −3 and 11.20, typically between 0.50 and 11.20, advantageously between 3 and 9.00. In some embodiments, the partition coefficient P ranges from −2 to 9. Or the degradable block copolymer crosslinker has a hydrophobic/hydrophilic balance R between 1 and 20, advantageously between 3 and 15.

The hydrophobicity of the crosslinker, and thus the partition coefficient P or the hydrophobic/hydrophilic balance R of the crosslinker, influences the loading of the microsphere in buprenorphine or salts thereof and then the release of the buprenorphine or salts thereof. A crosslinker having a partition coefficient P of more than 1.90 or a hydrophobic/hydrophilic balance R more than 2 leads to an optimal release of buprenorphine or salts thereof, in particular because it prevents the immediate release of a large part of the buprenorphine or salts thereof.

As used herein, the expression “copolymer cross-linker” means that the copolymer contains a functional group containing a double bond at least two of its extremities in order to link together several polymer chains.

The cross-linker c) as defined above is linear or star-shaped (advantageously from 3 to 8 arms) and it presents (CH₂═(CR₁₁))-groups at all its extremities (i.e. at its two extremities when linear and at the end of each arm when star-shaped), each R₁₁ being independently of one another hydrogen atom or a (C₁-C₆)alkyl group, preferably a methyl group. Advantageously, the crosslinker c) presents (CH₂═(CR₁₁))—CO— at all its extremities, each R₁₁ being independently of one another hydrogen atom or a (C₁-C₆)alkyl group, preferably a methyl group. Advantageously, all the R₁₁ are identical and are hydrogen atom or a (C₁-C₆)alkyl group, preferably a methyl group.

In some embodiments, the cross-linker c) as defined above is linear and it presents (CH₂═(CR₁₁))— groups at both its extremities, each R₁₁ being independently of one another hydrogen atom or a (C₁-C₆)alkyl group. Advantageously, the crosslinker c) presents (CH₂═(CR₁₁))—CO— groups at both its extremities, each R₁₁ being independently of one another hydrogen atom or a (C₁-C₆)alkyl group, preferably a methyl group. Advantageously, the R₁₁ are identical and are H or a (C₁-C₆)alkyl group, preferably a methyl group.

The crosslinker c) is of general formula (IIIa) or (IIIc) as follows:

(CH₂═CR₁₁)—CO—Xₙ-PEGₚ-Xₖ—CO—(CR₁₁═CH₂) (IIIa);

W(PEGₚ-Xₙ—O—CO—(CR₁₁═CH₂))_{z} (IIIc);

wherein:
each R₁₁ is independently of one another hydrogen atom or a (C₁-C₆)alkyl group;
X independently represents PLA, PGA, PLGA, PCL or PLAPCL;
n, k and p respectively represent the degree of polymerization of X, and PEG, n and k independently being integers from 1 to 150, and p being an integer from 1 to 100;
W is a carbon atom, a C₁-C₆-alkyl group (preferably a C₁-C₃-alkyl) or an ether group comprising 1 to 6 carbon atoms, preferably 1 to 3 carbon atoms;
z is an integer from 3 to 8.

Crosslinker c) of formula (IIIc) is a star-shaped polymer, i.e., a polymer consisting of several linear chains (also designated arms) connected a central core. In the crosslinker of formula (IIIc), W is the core of the star-shaped polymer and -(PEGₚ-Xₙ—O—CO—(CR₁₁═CH₂)) is an arm of the star-shaped polymer with z being the number of arms.

Advantageously, when the crosslinker c) is of general formula (IIIc), n may be identical or different in each arm of the PEG.

In the context of the invention, the abbreviations used herein have the following meaning:

| Abb. | Name | | Formula |
|---|---|---|---|
| PEG | polyethylene glycol | PEGₚ | 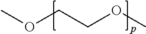 |
| PLA | poly-lactic acid (also named poly-lactide) | PLA_{n or k} | 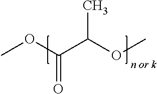 |
| PGA | poly-glycolic acid (also named poly-glycolide) | PGA_{n or k} | 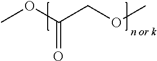 |
| PLGA | poly-lactic-glycolic acid The copolymer comprises both lactide and glycolide units, the degree of polymerization is the sum of the number of lactide and glycolide units | PLGA_{n or k} | 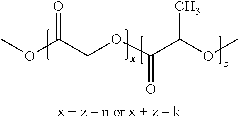 |
| PCL | poly(caprolactone) poly-lactic acid poly- caprolactone | PCL_{n or k} | 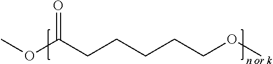 |
| PLAPCL | The copolymer comprises both lactide and caprolactone units, the degree of polymerization is the sum of the number of lactide and caprolactone units | PLAPCL_{n or k} | 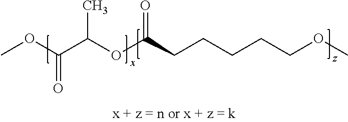 |

In the above table, n, p and k have the values disclosed herein.

In the above formula (IIIa), p is preferably an integer from 1 to 25, preferably from 2 to 15.

In the above formula (IIIc), p is preferably an integer from 1 to 16.

Advantageously, the crosslinker c) is of general formula (IIIa) or (IIIc), in particular (IIIa), as defined above, wherein X represents PLAPCL or PCL. More advantageously, the crosslinker c) is of general formula (IIIa) or (IIIc), in particular (IIIa) wherein X represents PCL.

Advantageously, the crosslinker c) is of general formula (IIIa) or (IIIc), in particular (IIIa), as defined above, wherein n and k independently are integers from 1 to 150, preferably from 1 to 20, more preferably from 1 to 10, even more preferably from 4 to 7. Preferably n+k ranges from 5 to 15 or from 8 to 14 and p is an integer from 1 to 100, preferably from 1 to 20.

Advantageously, the crosslinker c) is of general formula (IIIa) or (IIIc), in particular (IIIa) as defined above, wherein the R₁₁ are identical and are H or a (C₁-C₆)alkyl group.

Advantageously, the crosslinker c) is selected from the group consisting of compounds of general formula (IIIa) or (IIIc), in particular (IIIa), as defined above, wherein:
X=PLA, n+k=12 and p=13 (such as PEG₁₃-PLA₁₂ wherein R₁₁ is methyl);
X=PLAPCL, n+k=10 and p=13 (such as PEG₁₃-PLA₈-PCL₂ or PEG₁₃-PLA₇-PCL₃, wherein R₁₁ is methyl);
X=PLAPCL, n+k=9 and p=13 (such as PEG₁₃-PLA₄-PCL₅, wherein R₁₁ is methyl);
X=PLAPCL, n+k=8 and p=13 (such as PEG₁₃-PLA₂-PCL₆, wherein R₁₁ is methyl);
X=PCL; n+k=8 and p=13 (such as PEG₁₃-PCL₈, wherein R₁₁ is methyl);
X=PLGA; n+k=12 and p=13 (such as PEG₁₃-PLGA₁₂, wherein R₁₁ is methyl);
X=PCL, n+k=10 and p=4 (such as PEG₄-PCL₁₀, wherein R₁₁ is methyl); or
X=PCL, n+k=12 and p=2 (such as PEG₂-PCL₁₂, wherein R₁₁ is methyl).

In these embodiments, R₁₁ is preferably hydrogen or methyl.

In some embodiments, the crosslinker c) is a compound of general formula (IIIc), as defined above, wherein p is 7, X=PLAPCL, n=10, z is 3 with R₁₁ being preferably hydrogen or methyl (such as PEG 3-arm-PLA₇-PCL₃, wherein R₁₁ is methyl).

In some embodiments, the crosslinker c) is selected from the group consisting of compounds of general formula (IIIa) or (IIIc), in particular (IIIa), as defined above, wherein:
X=PLA, n+k=12 and p=13 (such as PEG₁₃-PLA₁₂, wherein R₁₁ is methyl);
X=PLAPCL, n+k=10 and p=13 (such as PEG₁₃-PLA₈-PCL₂ or PEG₁₃-PLA₇-PCL₃, wherein R₁₁ is methyl);
X=PLAPCL, n+k=9 and p=13 (such as PEG₁₃-PLA₄-PCL₅, wherein R₁₁ is methyl);
X=PLGA; n+k=12 and p=13 (such as PEG₁₃-PLGA₁₂, wherein R₁₁ is methyl);
X=PCL; n+k=8 and p=13 (such as PEG₁₃-PCL₈, wherein R₁₁ is methyl); or
X=PCL, n+k=12 and p=2 (such as PEG₂-PCL₁₂, wherein R₁₁ is methyl).

In these embodiments, R₁₁ is preferably hydrogen or methyl.

In some embodiments, the crosslinker c) is selected from the group consisting of compounds of general formula (IIIa), as defined above, wherein:
X=PLAPCL, n+k=10 and p=13 (such as PEG₁₃-PLA₇-PCL₃, wherein R₁₁ is methyl);
X=PLAPCL, n+k=9 and p=13 (such as PEG₁₃-PLA₄-PCL₅, wherein R₁₁ is methyl).

In these embodiments, R₁₁ is preferably hydrogen or methyl.

Within the definitions of the crosslinker c) above, the polyethylene glycol (PEG) has typically a number average molecular weight (Mn) of 100 to 10 000 g/mol, preferably 100 to 2 000 g/mol, more preferably 100 to 1 000 g/mol.

The amount of crosslinker c) typically ranges from 5 mol % to 90 mol %, preferably from 5 mol % to 60 mol %, relative to the total number of moles of components a), b) and c) (or relative to the total number of moles of components a), b), c) and e) when e) is present—see below).

The crosslinker c) is advantageously present in the reaction mixture in an amount ranging from 5 mol % to 90 mol %, preferably from 5 mol % to 60 mol %, relative to the total number of moles of components a), b) and c).

The crosslinked matrix of the hydrophilic degradable microsphere is advantageously further based on a chain transfer agent d), preferably results from the polymerization of components a), b) and c) in presence of a chain transfer agent d).

For the purposes of this invention, “transfer agent” means a chemical compound having at least one weak chemical bond. This agent reacts with the radical site of a growing polymer chain and interrupts the growth of the chain. In the chain transfer process, the radical is temporarily transferred to the transfer agent which restarts growth by transferring the radical to another polymer or monomer.

Advantageously, the chain transfer agent d) is selected from the group consisting of monofunctional or polyfunctional thiols, alkyl halides, transition metal salts or complexes and other compounds known to be active in free radical chain transfer processes such as 2,4-diphenyl-4-methyl-1-pentene. More advantageously, the chain transfer agent is a cycloaliphatic or aliphatic, thiol preferably having from 2 to 24 carbon atoms, more preferably between 2 and 12 carbon atoms, and having or not a further functional group selected from the groups amino, hydroxy and carboxy. Advantageously, the chain transfer agent d) is selected from the group consisting of thioglycolic acid, 2-mercaptoethanol, dodecane thiol and hexane thiol.

The amount of chain transfer agent d) typically ranges from 0.1 to 10 mol %, preferably from 2 to 5 mol %, relative to the number of moles of hydrophilic monomer a).

The chain transfer agent d) is advantageously present in the reaction mixture in an amount of, for example, from 0.1 to 10 mol %, preferably from 2 to 5 mol %, relative to the number of moles of hydrophilic monomer a).

In a particular aspect of the invention, the crosslinked matrix is only based on starting components a), b), c) and optionally d), as defined above and in the contents abovementioned, no other starting component are thus added to the reaction medium. It is thus clear that in such a case the sum of the above-mentioned contents of monomers (components (a), (b) and (c)) must be equal to 100%.

In some embodiments, the crosslinked matrix is advantageously further based on, preferably results from the polymerization of, at least a further ionised or ionisable monomer e) of general formula (V):

(CH₂═CR₁₂)-M-E (V),

wherein:
R₁₂ is hydrogen atom or a (C₁-C₆)alkyl group;
M is a single bond or a divalent radical having 1 to 20 carbon atoms, advantageously a single bond;
E is a ionised or ionisable group being advantageously selected from the group consisting of —COOH, —COO⁻, —SO₃H, —SO₃⁻, —PO₄H₂, —PO₄H⁻, —PO₄²⁻, —NR₁₃R₁₄, and —NR₁₅R₁₆R₁₇⁺; R₁₃, R₁₄, R₁₅, R₁₆ et R₁₇ being independently of one another hydrogen atom or a (C₁-C₆)alkyl group.

In the context of the invention, an ionised or ionisable group is understood to be a group which is charged or which may be in charged form (in the form of an ion), i.e. which carries at least one positive or negative charge, depending on the pH of the medium. For example, the COOH group may be ionised in the COO⁻ form, and the NH₂ group may be ionised in the form of NH₃⁺.

The introduction of an ionised or ionisable monomer into the reaction media makes it possible to increase the hydrophilicity of the resulting microspheres, thereby increasing the swelling rate of said microspheres, further facilitating their injection via catheters and microcatheters. In addition, the presence of an ionised or ionisable monomer improves the loading of active substances into the microsphere.

In an advantageous embodiment, the ionised or ionisable monomer e) is a cationic monomer, advantageously selected from the group consisting of 2-(methacryloyloxy)ethyl phosphorylcholine, 2-(dimethylamino)ethyl (meth)acrylate, 2-(diethylamino)ethyl (meth)acrylate and 2-((meth)acryloyloxy)ethyl] trimethylammonium chloride, more advantageously the cationic monomer is diethylaminoethyl (meth)acrylate. Advantageously, the ionised or ionisable e) is present in the reaction mixture in an amount of between 0% and 30% by mole, advantageously between 1% and 30% by mole, preferably from between 10% and 20% or 15% by mole, relative to the total number of moles of the monomers (components a)+b)+c)+e)). It is thus clear that in such a case the sum of the above-mentioned contents of monomers (components (a), (b) and (c) and (e)) must be equal to 100%.

In another advantageous embodiment, the ionised or ionisable monomer e) is an anionic monomer advantageously selected from the group consisting of acrylic acid, methacrylic acid, 2-carboxyethyl acrylate, 2-carboxyethyl acrylate oligomer, 3-sulfopropyl (meth)acrylate potassium salt and 2-[(methacryloyloxy)ethyl]dimethyl-(3-sulfopropyl)ammonium hydroxide, more advantageously, the anionic monomer is acrylic acid or methacrylic acid. The amount of ionised or ionisable monomer e) typically ranges from 0.1 to 30%, or from 1% to 25%, or from 1 to 10%, preferably from 2 to 5% by mole, relative to the total number of moles of the monomers (components a)+b)+c)+e)). It is thus clear that in such a case the sum of the above-mentioned contents of monomers (components (a), (b) and (c) and (e)) must be equal to 100%.

Advantageously, the ionised or ionisable monomer e) is present in the reaction mixture in an amount that ranges from 0% to 30 mol %, advantageously from 1 mol % to 30 mol %, preferably from 10 mol % to 15 mol %, relative to the total number of moles of the monomers ((components a)+b)+c)+e)). Advantageously, the ionised or ionisable monomer e) is acrylic acid and is advantageously present in the reaction mixture in an amount that ranges from 0% to 30% by mole, advantageously from 1% and 30% by mole, preferably from 10% to 15% by mole, relative to the total number of moles of the monomers (components a)+b)+c)+e)). It is thus clear that in such a case the sum of the above-mentioned contents of monomers (components (a), (b) and (c) and (e)) must be equal to 100%.

In some embodiments, the hydrophilic degradable microsphere comprises a crosslinked matrix that is based on at least, preferably that results from the polymerization of the following components:
a) from 10 to 90 mol % of a hydrophilic monomer of general formula (I):

(CH₂═CR₁)—CO-D (I)

wherein:
D is O—Z, Z being —(CH₂—CH₂—O)ₘ—H or —(CH₂—CH₂—O)ₘ—CH₃, with m being an integer from 1 to 30;
R₁ is hydrogen atom or a (C₁-C₆)alkyl group, preferably a methyl;
preferably m-PEGMA,
b) from 0.1 to 30 mol % of a cyclic monomer of formula (II):

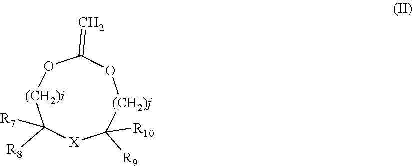

wherein:
R₇, R₈, R₉ and R₁₀ are, independently of one another, hydrogen atom or a (C₅-C₇)aryl group;
i and j are independently of one another an integer chosen between 0 and 1;
X is a single bond or an oxygen atom;
preferably 2-methylene-1,3-dioxepane;
   and
c) from 5 to 90 mol % of a degradable block copolymer cross-linker of formula:

(CH₂═CR₁₁)—CO—Xₙ-PEGₚ-Xₖ—CO—(CR₁₁═CH₂) (IIIa), or

W(PEGₚ-Xₙ—O—CO—(CR₁₁═CH₂))_{z} (IIIc);
wherein R₁₁, X, W, n, p, k, z are as disclosed herein,
preferably wherein
   R₁₁ is independently of one another hydrogen atom or a (C₁-C₆)alkyl group;
   X=PLA, n+k=12 and p=13 (such as PEG₁₃-PLA₁₂ wherein R₁₁ is methyl); or
   X=PLAPCL, n+k=10 and p=13 (such as PEG₁₃-PLA₈-PCL₂ or PEG₁₃-PLA₇-PCL₃, wherein R₁₁ is methyl); or
   X=PLAPCL, n+k=9 and p=13 (such as PEG₁₃-PLA₄-PCL₅, wherein R₁₁ is methyl); or
   X=PLAPCL, n+k=8 and p=13 (such as PEG₁₃-PLA₂-PCL₆, wherein R₁₁ is methyl); or
   X=PLGA; n+k=12 and p=13 (such as PEG₁₃-PLGA₁₂, wherein R₁₁ is methyl); or
   X=PCL; n+k=8 and p=13 (such as PEG₁₃-PCL₈, wherein R₁₁ is methyl); or
   X=PCL, n+k=10 and p=4 (such as PEG₄-PCL₁₀, wherein R₁₁ is methyl); or
   X=PCL, n+k=12 and p=2 (such as PEG₂-PCL₁₂, wherein R₁₁ is methyl);
and wherein the degradable block copolymer crosslinker has a partition coefficient P between −3 and 11.2, typically between 0.5 and 11.2 or a hydrophobic/hydrophilic balance R between 1 and 20;
   wherein mol % of components a) to c) are expressed relative to the total number of moles of compounds a), b) and c).

The amounts of components a), b) and c) may be as disclosed herein.

The microsphere of the invention can be readily synthesized by numerous methods well-known to the one skilled in the art. By way of example, the microsphere of the invention can be obtained by direct or inverse suspension polymerization as described below and, in the Examples, or by microfluidic.

A direct suspension may proceed as follows:

(1) stirring or agitating a mixture comprising
(i) at least the starting components a), b) and c) as defined above;
(ii) a polymerization initiator present in amounts ranging from 0.1 to approximately 2 parts per weight per 100 parts by weight of the monomers;
(iii) a surfactant in an amount no greater than about 5 parts by weight per 100 parts by weight of the aqueous solution, preferably no greater than about 3 parts by weight and most preferably in the range of 0.5 to 1.5 parts by weight;
(iv) a salt in an amount no greater than about 10 parts by weight per 100 parts by weight of the aqueous solution, preferably no greater than about 5 parts by weight and most preferably in the range of 1 to 4 parts by weight; and (iv) water to form an oil in water suspension;
   and
   (2) polymerizing the starting components.

In such a direct suspension polymerization, the surfactant may be selected from the group consisting of hydroxyethylcellulose, polyvinyl alcohol (PVA), polyvinylpyrrolidone, polyethylene oxide, polyethylene glycol and Polysorbate 20 (Tween® 20).

An inverse suspension may proceed as follows:

(1) stirring or agitating a mixture comprising:
(i) at least the starting components a), b) and c) as defined above;
(ii) a polymerization initiator present in amounts ranging from 0.1 to approximately 2 parts per weight per 100 parts by weight of the monomers;
(iii) a surfactant in an amount no greater than about 5 parts by weight per 100 parts by weight of the oil phase, preferably no greater than about 3 parts by weight and most preferably in the range of 0.5 to 1.5 parts by weight; and
(iv) oil to form a water in oil suspension;
   and
   (2) polymerizing the starting components.

In such a reverse suspension process, the surfactant may be selected from the group consisting of sorbitan esters such as sorbitan monolaurate (Span® 20), sorbitan monopalmitate (Span® 40), sorbitan monooleate (Span® 80), and sorbitan trioleate (Span® 85), hydroxyethyl cellulose, mixture of glyceryl stearate and PEG stearate (Arlacel®) and cellulose acetate.

In the above processes, the polymerization initiator may include t-butyl peroxide, benzoyl peroxide, azobiscyanovaleric acid (also known as 4,4′-azobis(4-cyanopentanoic acid)), AIBN (azobisisobutyronitrile), or 1,1′-azobis (cyclohexane carbonitrile) or one or more photo-initiators such as 2-hydroxy-4′-(2-hydroxyethoxy)-2-methylpropiophenone (106797-53-9); 2-hydroxy-2-methylpropiophenone (Darocur® 1173, 7473-98-5); 2,2-dimethoxy-2-phenylacetophenone (24650-42-8); 2,2-dimethoxy-2-phenyl acetophenone (Irgacure®, 24650-42-8) or 2-methyl-4′-(methylthio)-2-morpholinopropiophenone (Irgacure®, 71868-10-5).

Further, the oil may be selected from paraffin oil, silicone oil and organic solvents such as hexane, cyclohexane, ethyl acetate or butyl acetate.

Loading may proceed by numerous methods well-known to one of skill in the art such as passive adsorption (swelling of the polymer into a drug solution) or by ionic interaction.

In order to increase the drug loading and control the rate of drug release, a concept consists to introduce certain chemical moieties into the polymer backbone that are capable of interacting with the drug via non covalent interactions. Examples of such interactions include electrostatic interactions (described after), hydrophobic interactions, π-π stacking, and hydrogen bonding, among others.

### Drug

The composition comprises an effective amount of buprenorphine or a pharmaceutically acceptable salt thereof.

Advantageously, the buprenorphine is in the form of a free base or a pharmaceutically acceptable salt thereof such as buprenorphine hydrochloride. Advantageously, the buprenorphine is in the form of hydrochloride salt.

Advantageously, in the composition of the invention, the buprenorphine or the pharmaceutically acceptable salt thereof is loaded/absorbed onto the microsphere as defined above by non-covalent interactions. This particular way of entrapping drugs or prodrugs is called physical entrapment.

Loading of buprenorphine or the pharmaceutically acceptable salt thereof onto the microsphere of the invention may be proceeded by numerous methods well-known to the one skilled in the art such as preloading buprenorphine after the microsphere synthesis. Buprenorphine or the pharmaceutically acceptable salt thereof in solution is mixed with the degradable microspheres over a predetermined time and then the mixture is freeze-dried.

Advantageously, the composition of the invention comprises between 0.1 and 50 mg/mL of buprenorphine or a pharmaceutically acceptable salt thereof, more advantageously between 0.5 and 10 mg/mL.

Advantageously, the composition of the invention releases the buprenorphine or a pharmaceutically acceptable salt thereof without a burst, less 20 wt % of initial loading during the first day, followed by a constant delivery rate between 5 wt % and 15 wt % of initial loading every day for 1 to 30 days.

Advantageously, after implantation in living organisms, the composition of the invention releases the buprenorphine or a pharmaceutically acceptable salt thereof in the plasma without a burst during the first hour following implantation (skin, joint, viscera, muscle). Concentration of buprenorphine could remain in the therapeutic range for 1 to 7 days, advantageously for 1 to 15 days, preferably for 1 to 30 days, the therapeutic range being between 1 ng/mL to 8 ng/mL, or preferably without plasma detection when the loading value of buprenorphine is low (<1 mg/mL) for local delivery in order to obtain a peripheral analgesia without plasmatic exposition.

### Composition

The composition comprises an effective amount of buprenorphine or a pharmaceutically acceptable salt thereof, at least one hydrophilic degradable microsphere as defined above, and a pharmaceutically acceptable carrier. The carrier is suitable for administration by injection.

The buprenorphine or a pharmaceutically acceptable salt thereof and the hydrophilic degradable microsphere are as defined above.

According to the invention, the pharmaceutically acceptable carrier is intended for administration of the buprenorphine or a pharmaceutically acceptable salt thereof by injection and is advantageously selected in the group consisting in water for injection, starch, hydrogel, polyvinylpyrrolidone, polysaccharide, hyaluronic acid ester, contrast agent, and plasma.

The formulations may be administered by intramuscular, subcutaneous or intra-articular injection. The formulations of degradable microspheres are syrangable, the microsphere size and distribution are shown in Example 7 for example. This enables the administration in a needle that is from between 21 and 34 gauge.

The composition of the invention can also contain a buffering agent, a preservative, a gelling agent, a surfactant, or mixtures thereof. Advantageously, the pharmaceutically acceptable carrier is saline or water for injection.

The composition of the invention allows the extended-release, in particular the controlled-release, of buprenorphine or a pharmaceutically acceptable salt thereof over a period ranging from a few hours to a few months. Advantageously, the composition of the invention allows the controlled-release of buprenorphine or a pharmaceutically acceptable salt thereof for 1 day to 7 days, advantageously for 1 to 15 days, more advantageously for 1 to 30 days.

The composition of the invention allows the temporal control and the sustained-release as defined above, for example by modulating the nature and the contents of components a) and/or c).

The invention also relates to the composition as defined above, for use for preventing and/or treating moderate to severe pain.

The invention also relates to the composition as defined above, for use for preventing and/or treating post-surgical pain, in particular for locally treating post-surgical pain.

The invention also relates to the composition as defined above, for use for preventing and/or treating opioid use disorders such as opioid dependence, opioid tolerance and opioid withdrawal symptoms.

The invention also relates to a method for preventing and/or treating moderate to severe pain, comprising administering to a subject in need thereof an effective amount of the composition as defined above.

The invention also relates to a method for preventing and/or treating post-surgical pain, in particular for locally treating post-surgical pain, comprising administering, advantageously locally administering, to a subject in need thereof an effective amount of the composition as defined above.

The invention also relates to a method for preventing and/or treating opioid use disorders such as opioid dependence, opioid tolerance and opioid withdrawal symptoms, comprising administering to a subject in need thereof an effective amount of the composition as defined above.

The invention also relates to the use of the composition as defined above for the manufacturing of a drug for preventing and/or treating moderate to severe pain.

The invention also relates to the use of the composition as defined above for the manufacturing of a drug for preventing and/or treating post-surgical pain, in particular for locally treating post-surgical pain.

The invention also relates to the use of the composition as defined above for the manufacturing of a drug for preventing and/or treating opioid use disorders such as opioid dependence, opioid tolerance and opioid withdrawal symptoms.

### Extemporaneous Loading

In a particular embodiment of the invention, the buprenorphine or the pharmaceutically acceptable salt thereof may be loaded extemporaneously on dry and sterile microsphere.

The invention thus also relates to a pharmaceutical kit comprising:

i) at least one hydrophilic degradable microsphere as defined above in association with a pharmaceutically acceptable carrier for administration by injection;
ii) an effective amount of buprenorphine or a pharmaceutically acceptable salt thereof; and
iii) optionally an injection device, the hydrophilic degradable microsphere and the buprenorphine being packed separately.

In such an embodiment, the buprenorphine or the pharmaceutically acceptable salt thereof is advantageously intended to be loaded on the hydrophilic degradable microsphere just before the injection.

Advantageously, the buprenorphine or the pharmaceutically acceptable salt thereof is as defined above.

According to the present invention, “injection device” means any device for parenteral administration. Advantageously, the injection device is one or more syringes, which may be pre-filled, and/or one or more catheters or microcatheters.

### Use of the Microsphere

The invention also relates to the hydrophilic degradable microsphere as defined above for use for the delivery, advantageously the controlled-delivery, of an effective amount of buprenorphine or a pharmaceutically acceptable salt thereof to a subject in need thereof.

The buprenorphine or a pharmaceutically acceptable salt thereof and the hydrophilic degradable microsphere are as defined above.

Advantageously, the sustained delivery of buprenorphine or a pharmaceutically acceptable salt thereof is over a period ranging from a few hours to a few months without burst, advantageously from 1 day to 7 days, advantageously for 1 to 15 days, more advantageously for 1 to 30 days.

The invention also relates to a method for delivering an effective amount of buprenorphine or a pharmaceutically acceptable salt thereof to a subject in need thereof, advantageously over a period ranging from a few hours to a few months without burst, advantageously from 1 day to 7 days, advantageously for 1 to 15 days, more advantageously for 1 to 30 days, comprising the administration of the hydrophilic degradable microsphere as defined above in association with a pharmaceutically acceptable carrier for administration by injection.

The examples which follow illustrate the invention without limiting its scope in any way.

### EXAMPLES

“BPN” as used herein below stands for buprenorphine hydrochloride.

“MS” as used herein below stands for microsphere.

### Example 1: Preparation of Unloaded Microsphere According to the Invention

The starting components and their contents are summarized in Table 1a, Table 1b and table 1c. The tables summarize the main parameters used for MS synthesis.

TABLE 1a
Formulations of microspheres according to the invention for buprenorphine loading

| | | | Microspheres of 50-100 μm diameter | | | | | |
|---|---|---|---|---|---|---|---|---|
| | MS number | | MS1 | MS2 | MS3 | MS4 | MS5 | MS6 |
| Process | Oil/Water ratio (V/V) | | 1/11 | 1/11 | 1/11 | 1/11 | 1/11 | 1/11 |
| parameters | Stirring speed | | 240 RPM | 240 RPM | 240 RPM | 240 RPM | 240 RPM | 240 RPM |
| | PVA | | 1% | 1% | 1% | 1% | 1% | 1% |
| | NaCl | | 3% | 3% | 3% | 3% | 3% | 3% |
| Organic | Monomers mass/organic | | 56% | 56% | 56% | 56% | 56% | 56% |
| phase | phase mass (wt %) | | | | | | | |
| | Toluene (wt %) | | 44% | 44% | 44% | 44% | 44% | 44% |
| | Hexane thiol (component d) | | 3% | 3% | 3% | 3% | 3% | 3% |
| | (% mole/m-PEGMA mole) | | | | | | | |
| | AIBN (polymerization | | 0.28% | 0.28% | 0.28% | 0.28% | 0.28% | 0.28% |
| | initiator) | | | | | | | |
| | (% weight/organic phase | | | | | | | |
| | weight) | | | | | | | |
| | Phase | m-PEGMA | 85% | 85% | 85% | 85% | 85% | 85% |
| | monomer | (component a) | | | | | | |
| | | (% mole/total mole | | | | | | |
| | | monomer) | | | | | | |
| | | Crosslinker | 5% of PEG₁₃- | 5% of | 5% of | 5% of | 5% of | 5% of |
| | | (component c) | PLA₁₂ | PEG₁₃- | PEG₁₃- | PEG₁₃- | PEG₁₃- | PEG₂- |
| | | (% mole/total mole | | PLA₈- | PLA₇- | PLA₄- | PCL₈ | PCL₁₂ |
| | | monomer) | | PCL₂ | PCL₃ | PCL₅ | | |
| | | 2-methylene-1,3- | 10% | 10% | 10% | 10% | 10% | 10% |
| | | dioxepane (MDO) | | | | | | |
| | | (component b) | | | | | | |
| | | (% mole/total mole | | | | | | |
| | | monomer) | | | | | | |

TABLE 1b
Formulations of microspheres according to the invention for buprenorphine loading

| | | | Microspheres of 50-100 μm diameter | | | | | | |
|---|---|---|---|---|---|---|---|---|---|
| | MS number | | MS7 | MS8 | MS9 | MS10 | MS11 | MS12 | MS13 |
| Process | Oil/Water ratio (V/V) | | 1/11 | 1/11 | 1/11 | 1/11 | 1/11 | 1/11 | 1/11 |
| parameters | Stirring speed | | 240 RPM | 240 RPM | 240 RPM | 240 RPM | 240 RPM | 240 RPM | 240 RPM |
| | PVA | | 1% | 1% | 1% | 1% | 1% | 1% | 1% |
| | NaCl | | 3% | 3% | 3% | 3% | 3% | 3% | 3% |
| Organic | Monomers mass/ | | 56% | 56% | 56% | 56% | 56% | 56% | 56% |
| phase | organic phase mass | | | | | | | | |
| | (wt %) | | | | | | | | |
| | Toluene (wt %) | | 44% | 44% | 44% | 44% | 44% | 44% | 44% |
| | Hexanethiol | | 3% | 3% | 3% | 3% | 3% | 3% | 3% |
| | (component d) (% mole/ | | | | | | | | |
| | m-PEGMA mole) | | | | | | | | |
| | AIBN | | 0.28% | 0.28% | 0.28% | 0.28% | 0.28% | 0.28% | 0.28% |
| | (% weight/organic | | | | | | | | |
| | phase weight) | | | | | | | | |
| | Phase | m-PEGMA | 75% | 75% | 75% | 60% | 60% | 40% | 40% |
| | monomer | (component a) | | | | | | | |
| | | (% mole/total | | | | | | | |
| | | mole monomer) | | | | | | | |
| | | Crosslinker | 15% | 15% | 15% | 30% of | 30% of | 50% of | 50% |
| | | (component c) | of | of | of | PEG₁₃- | PEG₁₃- | PEG₁₃- | of |
| | | (% mole/total | PEG₁₃- | PEG₁₃- | PEG₂- | PLA₇-PCL₃ | PCL₈ | PCL₈ | PEG₂- |
| | | mole monomer) | PLA₇- | PCL₈ | PCL₁₂ | | | | PCL₁₂ |
| | | | PCL₃ | | | | | | |
| | | 2-methylene- | 10% | 10% | 10% | 10% | 10% | 10% | 10% |
| | | 1,3-dioxepane | | | | | | | |
| | | (MDO) | | | | | | | |
| | | (component b) | | | | | | | |
| | | (% mole/total | | | | | | | |
| | | mole monomer) | | | | | | | |

TABLE 1c
Formulations of microspheres according to the invention for buprenorphine loading

| | | | Microspheres of 50-100 μm diameter | | | |
|---|---|---|---|---|---|---|
| | MS number | | MS14 | MS15 | MS16 | MS17 |
| Process | Oil/Water ratio (V/V) | | 1/11 | 1/11 | 1/11 | 1/11 |
| parameters | Stirring speed | | 240 RPM | 240 RPM | 240 RPM | 240 RPM |
| | PVA | | 1% | 1% | 1% | 1% |
| | NaCl | | 3% | 3% | 3% | 3% |
| Organic | Monomers mass/organic phase | | 56% | 56% | 56% | 56% |
| phase | mass (wt %) | | | | | |
| | Toluene (wt %) | | 44% | 44% | 44% | 44% |
| | Hexanethiol (component d) | | 3 | 3 | 3 | 3 |
| | (% mole/m-PEGMA mole or | | | | | |
| | tert-butyl methacrylate) | | | | | |
| | AIBN (% weight/organic phase | | 0.28% | 0.28% | 0.28% | 0.28% |
| | weight) | | | | | |
| | Phase | m-PEGMA | 85% | 85% | 65% | 0% |
| | monomer | (component a) | | | | |
| | | (% mole/total mole | | | | |
| | | monomer) | | | | |
| | | Tert-butyl | 0% | 0% | 0% | 60% |
| | | methacrylate | | | | |
| | | (component a) | | | | |
| | | (% mole/total mole | | | | |
| | | monomer) | | | | |
| | | Crosslinker | 5% of | 5% PEG | 5% of | 30% of |
| | | (component c) | PEG₁₃- | 3-arm- | PEG₁₃-PLA₁₂ | PEG₁₃-PLA₇- |
| | | (% mole/total mole | PLGA₁₂ | PLA₇-PCL₃ * | | PCL₃ |
| | | monomer) | | | | |
| | | 2-methylene-1,3- | 10% | 10% | 10% | 10% |
| | | dioxepane (MDO) | | | | |
| | | (component b) | | | | |
| | | (% mole/total mole | | | | |
| | | monomer) | | | | |
| | | Methacrylic acid | 0% | 0% | 20% | 0% |
| | | (component e) | | | | |
| | | (% mole/total mole | | | | |
| | | monomer) | | | | |

* the crosslinker is 3 arm PEG with a molar mass of 1014 g/mol, PLA₇-PCL₃ as a total of 10 units.

The aqueous phase solution (917 mL) containing 1 wt % polyvinyl alcohol (Mw=13000-23000 g/mol), 3 wt % NaCl in deionized water was placed in a 1 dm³ reactor and heated up to 50° C.

The organic phase was prepared in an Erlenmeyer. Briefly, toluene (36.9 g) and 2,2′-azobis(2-methylpropionitrile) (AIBN) (0.28% weight/organic phase weight) were weighted. AIBN was introduced in another vial and solubilized in a volume fraction (z 30%) of the weighted toluene.

Then, degradable crosslinker was weighted in an Erlenmeyer. Polyethylene glycol methyl ether methacrylate (Mn=300 g/mol) or tert-butyl methacrylate (Mn=142.2 g/mol) and 2-methylene-1,3-dioxepane (MDO) was weighted and introduced into the Erlenmeyer. Then the remaining volume of toluene was been added to solubilize the monomers. Hexanethiol (3% mol/mol) of m-PEGMA or tert-butyl methacrylate) was added to the Erlenmeyer. The AIBN solution in toluene was added to the Erlenmeyer containing monomers. Finally, the organic phase had to be clear (monomer and initiator should be totally solubilized) without any aggregates before introduction into the aqueous phase.

The organic phase was poured into the aqueous phase at 50° C. Thereupon, stirring (240 rpm) was applied by using an impeller. After 4 minutes, the temperature had raised up to 80° C. After 8 hours, the stirring was stopped and microspheres were collected by filtration on a 40 μm sieve and washed extensively with acetone and water. Microspheres were then sieved with decreasing sizes of sieves (125, 100, 50 μm). MS in the size range 50-100 μm were collected for drug loading trials.

### Example 2: Loading of Microspheres According to Example 1 with Buprenorphine Hydrochloride (Preloading after MS Synthesis)

After the sieving step, 1 mL of microspheres obtained in example 1 (size range 50-100 μm) was placed in 15 mL polypropylene vials and 3 mL of sodium bicarbonate (4 mM) in water were added. The numbering of the MS batches according to their compositions is given in table 2. After 5 min of mixing with microspheres at room temperature, buprenorphine hydrochloride (Sigma, PHR1729-50MG) in water at 10 mg/mL was added (1 mg to 50 mg). Table 2 summarizes MS formulations used to investigate buprenorphine hydrochloride loading and sustained delivery.

TABLE 2
Crosslinker composition of degradable microspheres used for buprenorphine
hydrochloride loading

| | | | | | | | | | Tert- |
|---|---|---|---|---|---|---|---|---|---|
| | m-PEGMA | | | | | | | | butyl |
| | | | | | | | | PEG 3- | methacrylate |
| | | PEG₁₃- | PEG₁₃- | PEG₁₃- | | | | arm- | PEG₁₃- |
| Crosslinker | PEG₁₃- | PLA₈- | PLA₇- | PLA₄- | PEG₁₃- | PEG₂- | PEG₁₃- | PLA₇- | PLA₇- |
| % | PLA₁₂ | PCL₂ | PCL₃ | PCL₅ | PCLs | PCL₁₂ | PLGA₁₂ | PCL₃ | PCL₃ |
| 5 | ms1 εt | MS2 | MS3 | MS4 | MS5 | MS6 | MS14 | MS15 | |
| | MS16 | | | | | | | | |
| 15 | — | — | MS7 | — | MS8 | MS9 | | | |
| 30 | — | — | MS10 | — | MS11 | — | | | MS17 |
| 50 | — | — | — | — | MS12 | MS13 | | | |

The loading step was done at room temperature for 2 h under stirring on a tube rotator (≈30 rpm). Then, the supernatants were removed for the measurement of unbound buprenorphine hydrochloride by fluorimetry (λₑₓ 280 nm, λ_{Em} 354 nm). The amount of buprenorphine hydrochloride in supernatant was obtained by extrapolation from a standard curve (0.6 to 25 μg/mL). The loaded dose was calculated by subtracting the final amount of buprenorphine hydrochloride from the initial amount. The loading efficiency was calculated by the following equation: loading efficiency=((Buprenorphine hydrochloride in feed−Buprenorphine hydrochloride in supernatant)/Buprenorphine hydrochloride in feed)×100. The pellets were washed with 10 mL of water, then the microsphere pellets were frozen-dried before e-beam sterilization (15-25 kilograys). Table 3 summarizes buprenorphine loading for each MS formulation tested containing 5% of crosslinker.

TABLE 3
Buprenorphine hydrochloride loading on the microspheres containing
5% of degradable crosslinker according to example 1.

| | Buprenorphine | Buprenorphine | Efficacy of | Log P value for |
|---|---|---|---|---|
| MS | loading | loading target | buprenorphine | degradable |
| used | (mg/ml) | (mg/mL) | loading (%) | crosslinkers |
| MS1 | 0.85 ± 0.031 | 1 | 86 | 0.5 |
| MS2 | 0.88 ± 0.021 | 1 | 88 | 1.8 |
| MS3 | 0.82 ± 0.05 | 1 | 82 | 3.2 |
| MS3 | 1.59 ± 0.004 | 2 | 79 | 3.2 |
| MS3 | 3.03 ± 0.11 | 4 | 75 | 3.2 |
| MS4 | 0.79 ± 0.019 | 1 | 79 | 4.1 |
| MS4 | 1.63 ± 0.004 | 2 | 81 | 4.1 |
| MS4 | 3.11 ± 0.16 | 4 | 78 | 4.1 |
| MS5 | 0.94 ± 0.003 | 1 | 94 | 6.5 |
| MS5 | 42.7 ± 0.39 | 50 | 85 | 6.5 |
| MS6 | 0.97 ± 0.003 | 1 | 97 | 11.2 |
| MS14 | 0.89 ± 0.001 | 1 | 89 | 2.87 |
| MS15 | 0.96 ± 0.001 | 1 | 96 | 3.2 |

The partition coefficient P (Log P) values of degradable crosslinkers were determined in silico using Chemicalize provided by ChemAxon.
ND: not determined.

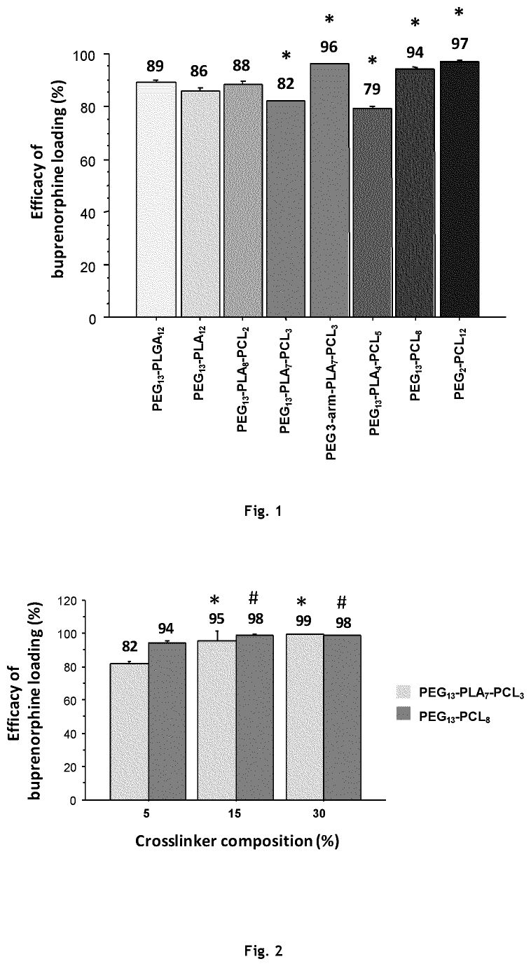

The loading of increasing amounts of buprenorphine hydrochloride was achievable with good yields on preformed microspheres. For the buprenorphine loading target of 1 mg/mL, the yield of drug loading for the different compositions of microspheres was 85% with small variations according to the composition of the crosslinker (Table 3 Et FIG. 1). The loading efficacy was significantly higher for the microspheres containing the most hydrophobic crosslinkers, PEG₁₃-PCL₈ and PEG₂-PCL₁₂ at 94% and 97%, respectively, and the loading yield was lower for MS containing the less hydrophobic crosslinkers (<90%) (Table 3 Et FIG. 1). Presence of a degradable tri-arm crosslinker PLA₇-PCL₃ at 5% instead of the linear one did not hinder the buprenorphine loading (MS15).

The effect of increasing crosslinker concentration incorporated in degradable microspheres was analyzed. Table 4 summarizes buprenorphine hydrochloride loading for MS formulation containing 5-15-30 or 50 mol % of degradable crosslinker. When the concentration of crosslinkers PEG₁₃-PLA₇-PCL₃ or PEG₁₃-PCL₈ increased (from 5% up to 30 mol %), the yield of buprenorphine hydrochloride was significantly improved (FIG. 2).

When microspheres contained 15%, 30% or 50 mol % of the degradable crosslinkers (PEG₁₃-PLA₇-PCL₃, PEG₁₃-PCL₈, PEG₂-PCL₁₂), the drug loading was almost complete, with yields higher than 95% for the loading objectives of 1 or 4 mg/mL (Table 4). No obvious difference exists for the loading of buprenorphine hydrochloride on microspheres containing the more hydrophobic crosslinkers, PEG₁₃-PCL₈ and PEG₂-PCL₁₂, the drug loading efficacy was higher than 95% for the loading target of 4 mg/mL. When the hydrophilic monomer m-PEGMA was replaced by the more hydrophobic tert-butyl methacrylate monomer, the loading of buprenorphine was achieved with a high efficiency (MS17).

TABLE 4
Effect of the crosslinker composition and concentration on buprenorphine loading
on the degradable microspheres.

| | | | | | | Log P value |
|---|---|---|---|---|---|---|
| | Buprenorphine | | % of | Buprenorphine | Efficacy of | for |
| MS | loading | Cross | degradable | loading target | buprenorphine | degradable |
| used | (mg/ml) | linker | crosslinker | (mg/mL) | loading (%) | crosslinkers |
| MS3 | 0.82 ± 0.01 | PEG₁₃- | 5 | 1 | 82 | 3.2 |
| MS7 | 0.95 ± 0.02 | PLA₇- | 15 | 1 | 95 | |
| MS10 | 0.99 ± 0.0007 | PCL₃ | 30 | 1 | 99 | |
| MS5 | 0.94 ± 0.003 | PEG₁₃- | 5 | 1 | 95 | 6.5 |
| MS5 | 42.7 ± 0.39 | PCL₈ | 5 | 50 | 85 | |
| MS8 | 0.98 ± 0.004 | | 15 | 1 | 98 | |
| MS8 | 3.87 ± 0.029 | | 15 | 4 | 97 | |
| MS8 | 43.4 ± 1 | | 15 | 50 | 86 | |
| MS11 | 0.98 ± 0.001 | | 30 | 1 | 98 | |
| MS12 | 3.90 ± 0.001 | | 50 | 4 | 96 | |
| MS6 | 0.97 ± 0.003 | PEG₂- | 5 | 1 | 97 | 11.2 |
| MS9 | 3.91 ± 0.013 | PCL₁₂ | 15 | 4 | 97 | |
| MS13 | 3.92 ± 0.025 | | 50 | 4 | 98 | |
| MS17 | 0.99 ± 0.001 | PEG₁₃- | 30 | 1 | 99 | 3.2 |
| | | PLA₇- | | | | |
| | | PCL₃ | | | | |

The partition coefficient P (Log P) values of degradable crosslinkers were determined in silico using Chemicalize provided by ChemAxon.

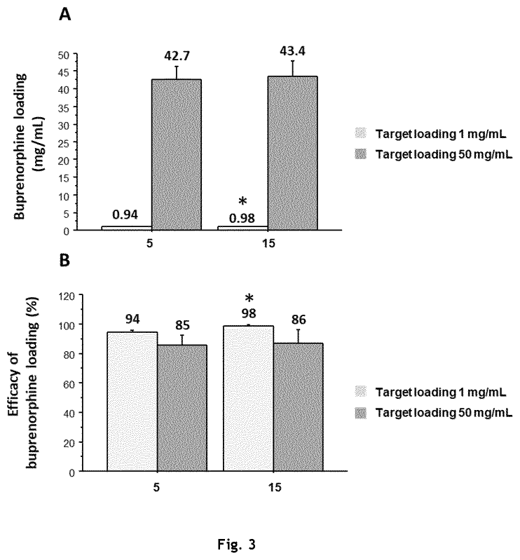

For the high loading objective of buprenorphine hydrochloride at 50 mg/mL on microspheres containing 5% or 15 mol % of crosslinker PEG₁₃-PCL₈, the loading efficacy was lower (85%) than for the objective of 1 mg/mL (>95%) (FIG. 3). Around 40 mg of buprenorphine are loadable on degradable microspheres containing 5% or 15 mol % of degradable crosslinker. The crosslinker content had no effect on efficacy of buprenorphine loading.

The payload of buprenorphine is tunable on preformed degradable microspheres. A low drug payload is indicated for the local release of low amounts of buprenorphine, for example to get a peripheral analgesia while a high drug loading could be useful to obtain constant plasmatic buprenorphine concentration for several weeks, for example to cure patients for opioids dependence.

### Example 3: Study of the In Vitro Release of Buprenorphine from Loaded Microspheres According to Example 2

For sterile and dry microspheres loaded with buprenorphine hydrochloride, the swelling step of microspheres was performed for 10 min in 10 mL of water (Sigma W-3500). After the removal of water, 25 mL of PBS (Sigma P-5368; 10 mM phosphate buffered saline; NaCl 0,136 M; KCl 0.0027 M; pH 7.4) were added.

Drug elution occurred at 37° C. under shaking (150 rpm), the tubes were placed horizontally in the oven. Samples (2 mL) were withdrawn after 5 h and every day. At each sampling time, the medium was completely renewed with fresh PBS. The amounts of free buprenorphine in water and in PBS supernatants were determined by fluorometry ((λₑₓ 280 nm, λ_{Em} 354 nm).

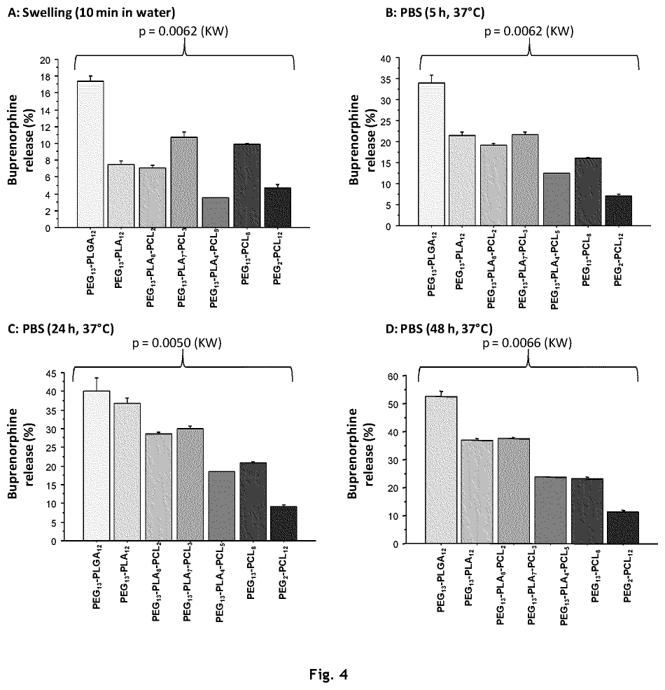

Results are given in Table 5 and FIGS. 4, 5, 6, 7 and 8.

Hydration of the sterile and dry microspheres loaded with buprenorphine was done in water for 10 min followed by a transfer in PBS. The amount of buprenorphine eluted during the hydration of microspheres is shown in Table 5 and in FIG. 4. A limited release of buprenorphine (<10%) with a significant effect of the crosslinker hydrophobicity was observed in water, except for MS14 where 17% of the payload was removed during the hydration step. The transfer of hydrated microspheres into PBS increased the amount of buprenorphine released, the drug elution decreased depending on the hydrophobicity of the degradable crosslinker.

The initial drug release was lower in presence of the more hydrophobic crosslinkers (PEG₁₃-PCL₈ and PEG₂-PCL₁₂), i.e. 10% at 48 h for MS6 compared to 35% for MS2 and MS3 which contained less hydrophobic crosslinkers (FIG. 4D). The in vitro release of buprenorphine in PBS after MS hydration in water depends on the hydrophobicity of degradable crosslinkers.

TABLE 5
In vitro analysis of buprenorphine burst release during swelling of dry microspheres in
water for 10 minutes and subsequent incubation in PBS for 5 h (37° C., 150 rpm).

| | | | | MS in vitro | |
|---|---|---|---|---|---|
| | Buprenorphine | % of buprenorphine release | | degradation | |
| MS | loading target | During MS | After 5 h | time | |
| used | (mg/mL) | swelling | in PBS | (days) | |
| MS1 | 1 | 7.5 | 21.4 | 4 | |
| MS2 | 1 | 7 | 19 | 7 | |
| MS3 | 1 | 9.7 | 20.4 | 10 | |
| MS3 | 2 | 5.7 | 17 | 10 | |
| MS3 | 4 | 8 | 21 | 10 | |
| MS4 | 1 | 3.5 | 12.7 | 50 | |
| MS4 | 2 | 4.6 | 11.3 | 50 | |
| MS4 | 4 | 7.5 | 15.1 | 50 | |
| MS5 | 1 | 9.9 | 16 | >6 | months |
| MS5 | 50 | 6.6 | 12 | >6 | months |
| MS6 | 1 | 4.6 | 7 | >6 | months |
| MS7 | 1 | 1.9 | 6.3 | >6 | months |
| MS8 | 1 | 1.2 | 3.4 | >6 | months |
| MS8 | 4 | 4.7 | 5.5 | >6 | months |
| MS8 | 50 | 4.6 | 6.5 | >6 | months |
| MS9 | 4 | 3.4 | 3.4 | >6 | months |
| MS10 | 1 | 1.05 | 6.1 | >6 | months |
| MS11 | 1 | 1.3 | 3.2 | >6 | months |
| MS12 | 4 | 3.4 | 5.1 | >6 | months |
| MS13 | 4 | 9.8 | 10.6 | >6 | months |
| MS14 | 1 | 17.3 | 34 | 1 | day |
| MS15 | 1 | 5.3 | 30.8 | 30 | days |
| MSV | 1 | 0 | 1.8 | >6 | months |

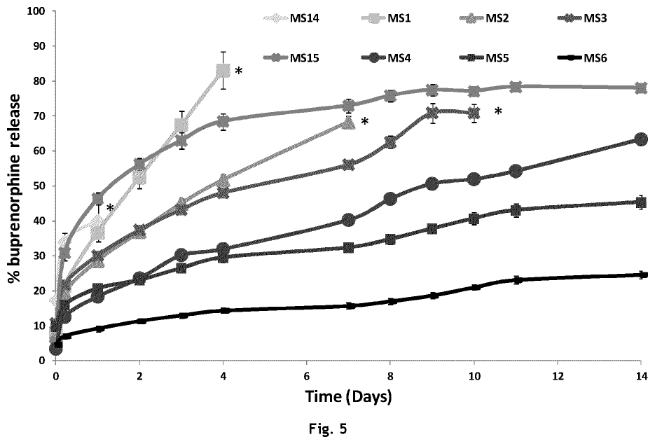

After the initial step of release in PBS (FIG. 4), the release profiles from the different microspheres were variable according to their degradation time which depends on the crosslinker composition (FIG. 5). In PBS, MS14, MS1, MS2, MS3 was degraded in 1, 4, 7 and 10 days respectively, while for MS containing more hydrophobic crosslinkers (PEG₁₃-PLA₄-PCL₅, PEG₁₃-PCL₈ and PEG₂-PCL₁₂), the degradation of MS was slower and was not achieved in 14 days. A long term and sustained delivery of buprenorphine could be achieved with MS6.

Crosslinker hydrophobicity controls the initial flow rate of buprenorphine from MS after the swelling step in water and the further steps of release by controlling the MS degradation time. The flow rate of buprenorphine from degradable microspheres of example 1 depends on the MS degradation time, and consequently the hydrophobicity of the crosslinker.

The duration of buprenorphine release in PBS increase with the MS degradation time, i.e. according to the partition coefficient P of the degradable crosslinker. The order of MS degradation during the release experiments follows the hydrophobicity of the crosslinkers, thus MS14 was degraded in 24 h, MS1 in 4 days, MS2 in 7 days, and MS3 in 10 days. The other MS (MS4, MS5 and MS6) with 5 mol % of more hydrophobic crosslinkers were not degraded during the 2 weeks of incubation in PBS. MS15 at 5 mol % of the tri-arm crosslinker degrade more slowly than MS3 at 5 mol % of the linear one, the release of buprenorphine was different between the 2 microspheres. The structure of the crosslinker (linear or branched) appears to be a factor regulating the release of buprenorphine from degradable MS.

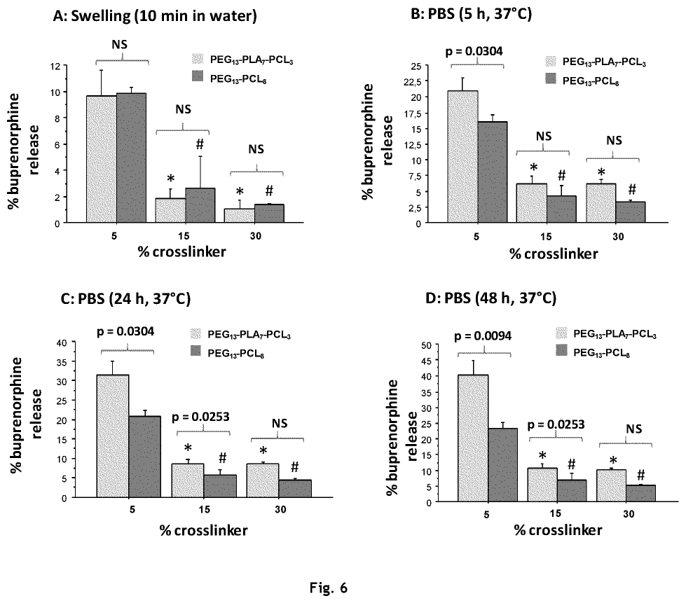

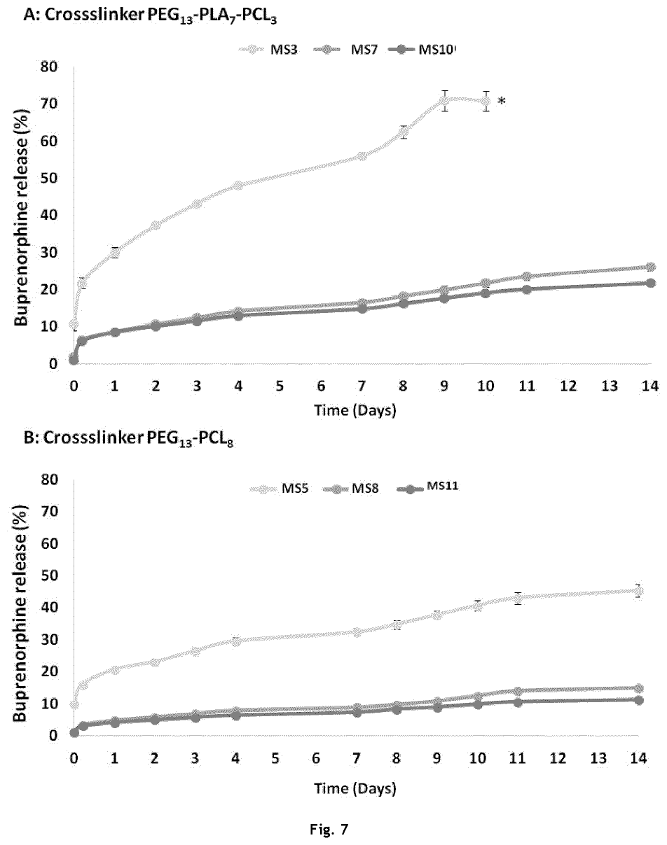

The effect of crosslinker concentration in degradable MS on the buprenorphine release is shown in FIGS. 6 and 7. Compared to the microspheres at 5 mol % of the crosslinker PEG₁₃-PLA₇-PCL₃ or PEG₁₃-PCL₈, the release of buprenorphine during the swelling step in water and the early times of incubation in PBS (5 h, 24 h and 48 h) was reduced significantly when the microspheres contain 15% or 30 mol % of crosslinker (FIG. 6). After 48 h in PBS, around 10% of buprenorphine was eluted from the highly crosslinked microspheres (MS7, MS10, MS8, MS11) compared to 40% or 20% for MS3 and MS5, respectively, which contain 5 mol % of degradable crosslinker of increasing hydrophobicity.

At high concentration of crosslinker, the elution of buprenorphine was significantly reduced compared to the microspheres at 5 mol % of crosslinker. Few differences exist between the 2 groups of crosslinkers at 15% or 30 mol % on the elution profiles of buprenorphine (FIGS. 6 and 7). The buprenorphine elution from degradable MS could be controlled by the crosslinking degree of the microsphere, showing a major effect of the hydrogel mesh size on the drug release.

The release of buprenorphine from the highly crosslinked microspheres (>5%) was sustained in PBS for at least 2 weeks. The rise of crosslinker concentration increased the duration of buprenorphine release and reduced the buprenorphine flow (FIG. 7).

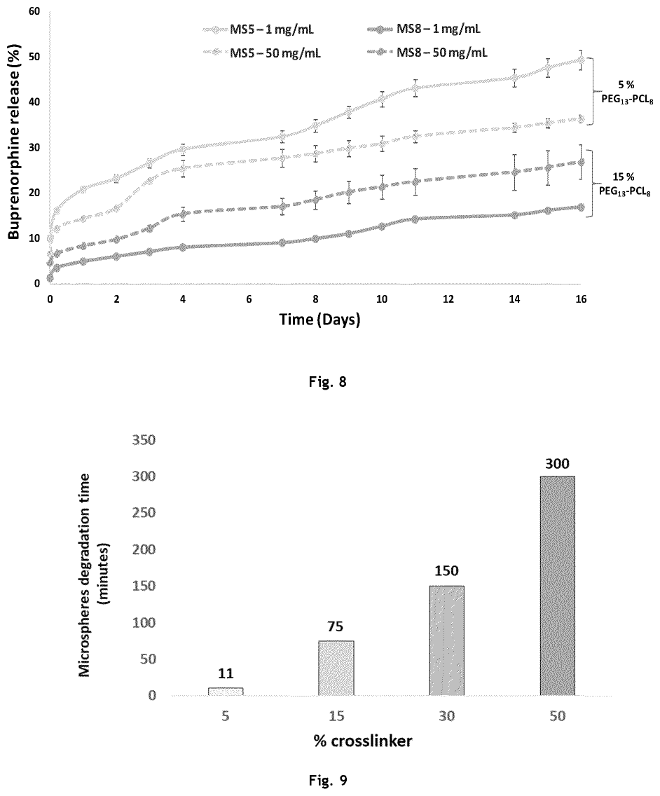

The effect of buprenorphine payload on the in vitro release profile was examined for 2 concentrations of the same crosslinker (FIG. 8). The buprenorphine release from degradable MS5 and MS8 microspheres was not profoundly altered by the payload: 1 mg/mL or 43 mg/mL (FIG. 8).

In accordance with previous observations (FIG. 7), the release for each payload depends on the crosslinker content, the release was lower for each payload for the MS containing 15 mol % of crosslinker (MS8) compared to MS containing 5 mol % of crosslinker (MS5). These results confirm that the hydrogel mesh size is an important parameter for the control of buprenorphine elution in addition to the crosslinker hydrophobicity.

The extension of duration of buprenorphine release could be explained by an increase of microspheres degradation time as observed during accelerated degradation experiment of microspheres in diluted sodium hydroxide (FIG. 9).

### Example 4. Extemporaneous Loading of Buprenorphine Hydrochloride on Sterile Microspheres According to Example 1

After the sieving step, a suspension of 1 mL of microspheres obtained in example 1 (size range 50-100 μm) in 15 mL of a solution containing 2.5% (w/v) of mannitol and 4 mM of sodium bicarbonate was prepared. After homogenization, the pellet of microspheres was recovered, frozen-dried and sterilized by e-beam radiation (15-25 kilograys). Then, buprenorphine hydrochloride (1 mg up to 4 mg) in solution in 3 mL of water was added to the sterile and dry microspheres.

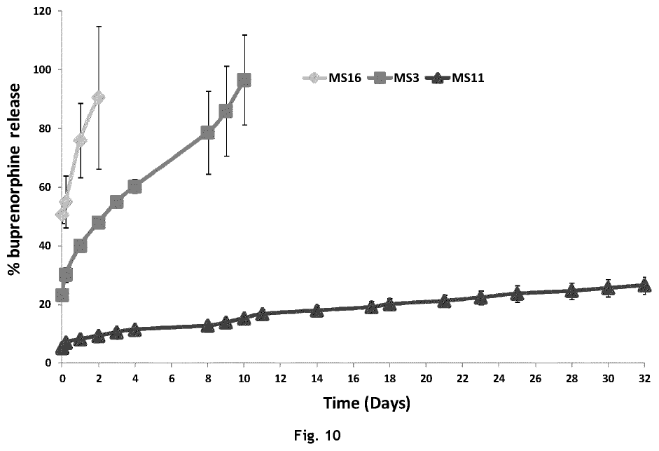

The loading step was done at room temperature for 5 minutes under stirring on a tube rotator (z 30 rpm). Then, the supernatants were removed for the measurement of unbound buprenorphine by fluorimetry (λₑₓ 280 nm, λ_{Em} 354 nm). The amount of buprenorphine hydrochloride was obtained by extrapolation from a standard curve (0.6 to 20 μg/mL). Table 6 summarizes extemporaneous buprenorphine loading on dry and sterile MS. Then, PBS (25 mL) was added to the microspheres loaded with buprenorphine hydrochloride for the in vitro drug release experiment (37° C., 150 rpm). The release of buprenorphine hydrochloride was analysed by fluorimetry (λₑₓ 280 nm, λ_{Em} 354 nm) (FIG. 10).

TABLE 6
Extemporaneous buprenorphine hydrochloride
loading on dry and sterile microspheres

| | Buprenorphine | Buprenorphine | Efficacy of |
|---|---|---|---|
| | loading target | loading | buprenorphine |
| MS used | (mg/mL) | (mg/mL) | loading (%) |
| MS3 | 1 | 0.89 ± 0.031 | 89 |
| MS3 | 2 | 1.18 ± 0.66 | 59 |
| MS3 | 4 | 2.16 ± 1.14 | 54 |
| MS11 | 4 | 2.56 ± 0.15 | 64 |
| MS16 | 1 | 0.84 ± 0.03 | 89 |

The loading of buprenorphine on dry and sterile microspheres was feasible with a mean yield of 71%. After the loading step, the immediate transfer of the microspheres in PBS induced a significant drug release for microspheres containing methacrylic acid (MS16) (FIG. 10). On contrary, for MS without methacrylic acid (MS3 Et MS11) the release was more sustained. The release depends on the MS degradation time, for MS3 at 5 mol % of a low hydrophobic crosslinker (PEG₁₃-PLA₇-PCL₃; Log P=3.2) the drug release lasted up to complete MS degradation in 10 days. For MS11 at 30 mol % of a more hydrophobic crosslinker (PEG₁₃-PCL₈; Log P=6.5), the flow rate of buprenorphine was lower and the drug release was not achieved after 1 month in PBS. An extemporaneous loading of buprenorphine on sterile and dry microspheres allows a sustained drug release controlled by the MS degradation time.

### Example 5: Study of the Plasma Buprenorphine Concentration During the Time Following Single Subcutaneous Injection in Rabbit of Degradable Microspheres Loaded with Buprenorphine According to Example 2

Degradable microspheres (MS3 Et MS4, duration buprenorphine elution of 10 and 30 days, respectively) loaded with buprenorphine (target loading of 2 or 4 mg/mL) were implanted subcutaneously in the dorsum of New-Zealand rabbit (n=5) without anesthesia to better observe the occurrence of a possible sedative effect. Injectable buprenorphine (Bupaq®, Multidose 0.3 mg/mL Injectable solution injectable, Virbac) was administrated at the dose of 60 μg/kg/day as reference. Blood samples (˜2.5 mL) were recovered at the auricular artery to assess the systemic concentration of buprenorphine at the following time points: pre-injection (T0), 15 min, 30 min, 1 h, 3 h, 5 h, 24 h, 2 d, 4 d, 7 d, 14 d, 21 d, 28 d. Buprenorphine quantifications were done by coupled HPLC-MS/MS (LOD=0.1 ng/mL, LOQ=0.3 ng/mL). The dose of buprenorphine injected was determined as the dose equivalent to repeated injectable buprenorphine injections over the period of buprenorphine elution form degradable microspheres, i.e. for a delivery of 10 days, the total dose injected with injectable buprenorphine would be 30 or 60 μg/kg per injection×10 injections (Table 7).

TABLE 7
Animal groups for pharmacokinetics study after a single subcutaneous injection of
microspheres loaded with buprenorphine.

| | | | Duration of in | Time | Daily | |
|---|---|---|---|---|---|---|
| | | Buprenorphine | vitro | equivalent | equivalence | Total dose of |
| | MS | loading | buprenorphine | treatment | buprenorphine | buprenorphine |
| Animals | used | (mg/mL) | release (days) | (days) | (μg/kg/Day) | injected (mg) |
| 1 | MS4 | 6.5* | 30 | 30 | 60 | 6.7 |
| 2 | MS4 | 3 | 30 | 10 | 60 | 2.2 |
| 3 | MS3 | 2.8 | 10 | 10 | 60 | 2.1 |
| 4 | MS3 | 3 | 10 | 10 | 60 | 2.5 |
| 5 | MS3 | 1.5 | 10 | 10 | 30 | 1.3 |

*pool of 2 pellets loaded at 3.2 and 3.3 mg/mL

The different doses administered to rabbits were chosen to allow for comparison of the animals by pairs with one parameter varying between the 2 animals: according to the MS degradation time and the amount of buprenorphine injected. Animals 1 and 2 received MS4 loaded at 3 mg/mL, but animal 1 received three times as much MS compared to animal 2, equivalent to a duration of treatment of 30 days and 10 days, respectively. Animals 2 and 3 received a similar amount of MS loaded at the same concentration, but animal 2 received MS4 while animal 3 received MS3. Animals 3 and 4 received the same treatment. Animals 4 and 5 both received MS3, but animal 4 received MS at 3 mg/mL and animal 5 received MS loaded at 1.5 mg/mL. The plasmatic levels were analyzed according to the MS degradation time and the buprenorphine dose (FIG. 11). As control, injectable buprenorphine (Bupac, 0.3 mg/mL) was administered once at 60 μg/Kg.

In vivo, after subcutaneous implantation in rabbit of buprenorphine loaded MS, the plasmatic passage of buprenorphine occurred without burst and lasted for 4 or 7 days for the MS3 and MS4, respectively, showing an effect of microsphere degradation time on in vivo buprenorphine elution (Table 8).

TABLE 8
Summary of selected pharmacokinetic values for sustained-release
buprenorphine administered subcutaneously to rabbit.

| | T ₘₐₓ | | T ₗₐₛₜ |
|---|---|---|---|
| Free buprenorphine | 30 | min | 5 h |
| MS3 | 5 | h | 96 h |
| MS4 | 24 | h | 168 h |

Increasing the amount of buprenorphine implanted for MS3 and MS4 did not extend the duration of plasma presence, but only the plasma concentration of buprenorphine rise (FIG. 11). The release of buprenorphine was accelerated in vivo compared to the in vitro tests (FIG. 5), where buprenorphine was eluted in PBS in 10 days from MS3, while for MS4 elution was not complete after 2 weeks in PBS. After subcutaneous implantation in rabbit, the buprenorphine release was delayed in plasma showing that degradable MS of example 1 are efficient buprenorphine delivery platform for several days. Implantation of MS with longer degradation times (MS5 or MS6) would allow release of buprenorphine for a longer period in plasma.

### Example 6: Tissular Concentration of Buprenorphine at the Implantation Site One Month after Injection of Buprenorphine Loaded Microspheres

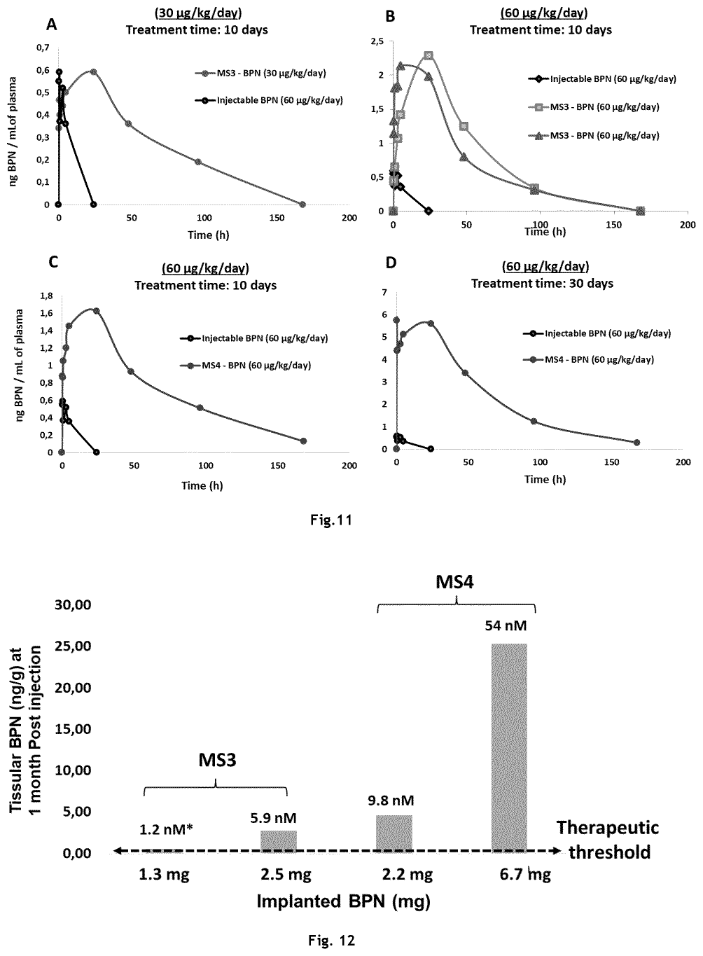

One month after implantation of MS3 and MS4, residual buprenorphine at implantation site was measured for all animals. In tissues, ng of buprenorphine per gram was secondary expressed in concentration (nM) (FIG. 12).

Buprenorphine was detected in the skin of injection sites one month after injection. The residual concentration in tissue depends of the microsphere degradation time and the amount of implanted drug. Otherwise, the residual local concentrations, between 1.2 and 54 nM, could still be active to inhibit locally the mu receptors of buprenorphine (EC₅₀ value of stimulation concentration for mu-receptor is around 1 nM) (Lutfy K, Cowan A. Curr Neuropharmacol 2004; 2: 395-402).

### Example 7: Size Distribution of Sterile Hydrophilic Microspheres (MS3 Et MS4) after Swelling in Saline

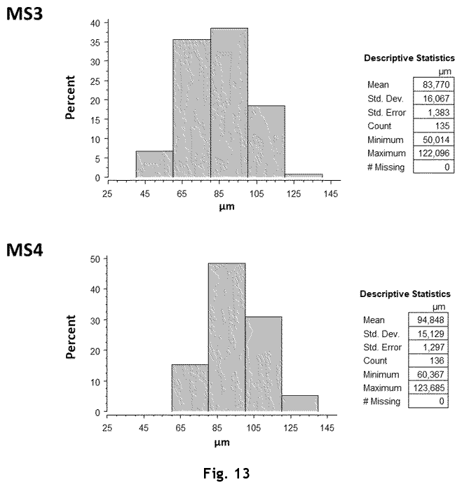

The sterile microspheres from the two batches used for the pharmacokinetic study have similar size distribution (50-120 μm) (FIG. 13).

1. A composition comprising an effective amount of buprenorphine or a pharmaceutically acceptable salt thereof, at least one hydrophilic degradable microsphere comprising a crosslinked matrix, and a pharmaceutically acceptable carrier for administration by injection, the crosslinked matrix being based on at least the following components:
a) from 10 mol % to 90 mol % of a hydrophilic monomer of general formula (I):
(CH₂═CR₁)—CO-D (I)
wherein:
D is O—Z or NH—Z, with Z being —(CR₂R₃)ₘ—CH₃, —(CH₂—CH₂—O)ₘ—H, (CH₂—CH₂—O)ₘ—CH₃, —(CR₂R₃)ₘ—OH or —(CH₂)ₘ—NR₅R₆ with m being an integer from 1 to 30;
R₁, R₂, R₃, R₄, R₅ and R₆ are, independently of one another, hydrogen atom or a (C₁-C₆)alkyl group;
b) from 0.1 mol % to 30 mol % of a cyclic monomer of formula (II): 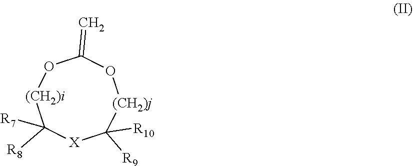
wherein:
R₇, R₈, R₉ and R₁₀ are, independently of one another, a hydrogen atom, a (C₁-C₆)alkyl group or an aryl group;
i and j are independently of one another an integer chosen between 0 and 2; and
X is a single bond or an oxygen atom;
and
c) from 5 mol % to 90 mol % of a linear or star-shaped degradable block copolymer cross-linker having a partition coefficient P of between −3 and 11.20, or a hydrophobic/hydrophilic balance R between 1 and 20, said degradable block copolymer cross-linker having the formula:
(CH₂═CR₁₁)—CO—Xₙ-PEGₚ-Xₖ—CO—(CR₁₁═CH₂) (IIIa); or
W(PEGₚ-Xₙ—O—CO—(CR₁₁═CH₂))_{z} (IIIc);
wherein
R₁₁ is independently a hydrogen atom or a (C₁-C₆)alkyl group;
X is independently PLA, PGA, PLGA, PCL or PLAPCL;
n and k are independently integers from 1 to 150;
p is an integer from 1 to 100;
W is a carbon atom, a C₁-C₆-alkyl group or an ether group comprising 1 to 6 carbon atoms;
z is an integer from 3 to 8;
wherein mol % of components a) to c) are expressed relative to the total number of moles of compounds a), b) and c).

2. The composition of claim 1, wherein the degradable block copolymer cross-linker c) is selected from the group consisting of compounds of general formula (IIIa) or (IIIc), wherein:
X=PLA, n+k=12 and p=13; or
X=PLAPCL, n+k=10 and p=13; or
X=PLAPCL, n+k=9 and p=13; or
X=PLAPCL, n+k=8 and p=13; or
X=PCL; n+k=8 and p=13; or
X=PLGA; n+k=12 and p=13; or
X=PCL, n+k=10 and p=4; or
X=PCL, n+k=12 and p=2.

3. The composition of claim 1, wherein the degradable block copolymer cross-linker c) is of general formula (IIIa) or (IIIc) wherein X represents PCL or PLAPCL.

4. The composition of claim 1, wherein the amount of degradable block copolymer cross-linker c) ranges from 5 mol % to 60 mol % relative to the total number of mole components a), b) and c).

5. The composition of claim 1, wherein the cyclic monomer b) is selected from the group consisting of 2-methylene-1,3-dioxolane, 2-methylene-1,3-dioxane, 2-methylene-1,3-dioxepane, 2-methylene-4-phenyl-1,3-dioxolane, 2-methylene-1,3,6-trioxocane and 5,6-benzo-2-methylene-1,3dioxepane.

6. The composition of claim 1, wherein the hydrophilic monomer a) is selected from the group consisting of sec-butyl acrylate, n-butyl acrylate, t-butyl acrylate, t-butyl methacrylate, methylmethacrylate, N-dimethyl-aminoethyl(methyl)acrylate, N,N-dimethylaminopropyl-(meth)acrylate, t-butylaminoethyl (methyl)acrylate, N,N-diethylaminoacrylate, acrylate terminated poly(ethylene oxide), methacrylate terminated poly(ethylene oxide), methoxy poly(ethylene oxide) methacrylate, butoxy poly(ethylene oxide) methacrylate, acrylate terminated poly(ethylene glycol), methacrylate terminated poly(ethylene glycol), methoxy poly(ethylene glycol) methacrylate, butoxy poly(ethylene glycol) methacrylate.

7. The composition of claim 1, wherein the crosslinked matrix of the hydrophilic degradable microsphere is further based on a chain transfer agent d).

8. The composition of claim 1, comprising from 0.1 to 50 mg/ml of buprenorphine or a pharmaceutically acceptable salt thereof.

9. The composition of claim 1 wherein the block copolymer cross-linker c) is a compound of general formula (IIIc) wherein p is 7, X=PLAPCL, n=10 and z is 3.

10. A method for preventing and/or treating moderate to severe pain or post-surgical pain, which comprises the step of administering a composition as defined in claim 1 to a subject in need thereof.

11. (canceled)

12. A method for preventing and/or treating opioid use disorders, which comprises the step of administering a composition as defined in claim 1 to a subject in need thereof.

13. The method according to claim 11, wherein the buprenorphine is released in a constant rate during a period of 1 day to 30 days.

14. A hydrophilic degradable microsphere for use for delivering an effective amount of buprenorphine or a pharmaceutically acceptable salt thereof to a subject in need thereof, the hydrophilic degradable microsphere comprising a crosslinked matrix, the crosslinked matrix being based on at least the following components:
a) from 10 mol % to 90 mol % of a hydrophilic monomer of general formula (I):
(CH₂═CR₁)—CO-D (I)
wherein:
D is O—Z or NH—Z, with Z being —(CR₂R₃)ₘ—CH₃, —(CH₂—CH₂—O)ₘ—H, (CH₂—CH₂—O)ₘ—CH₃, —(CR₂R₃)ₘ—OH or —(CH₂)ₘ—NR₅R₆ with m being an integer from 1 to 30;
R₁, R₂, R₃, R₄, R₅ and R₆ are, independently of one another, hydrogen atom or a (C₁-C₆)alkyl group;
b) from 0.1 mol % to 30 mol % of a cyclic monomer of formula (II): 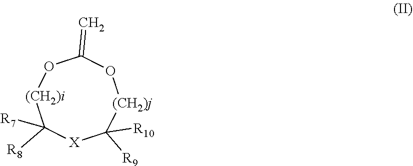
wherein:
R₇, R₈, R₉ and R₁₀ are, independently of one another, a hydrogen atom, a (C₁-C₆)alkyl group or an aryl group;
i and j are independently of one another an integer chosen between 0 and 2; and
X is a single bond or an oxygen atom;
and
c) from 5 mol % to 90 mol % of a linear or star-shaped degradable block copolymer cross-linker having a partition coefficient P of between −3 and 11.20, or a hydrophobic/hydrophilic balance R between 1 and 20, said degradable block copolymer cross-linker having the formula:
(CH₂═CR₁₁)—CO—Xₙ-PEGₙ-Xₖ—CO—(CR₁₁═CH₂) (IIIa); or
W(PEGₙ-Xₙ—O—CO—(CR₁₁═CH₂))_{z} (IIIc);
wherein
R₁₁ is independently a hydrogen atom or a (C₁-C₆)alkyl group;
X is independently PLA, PGA, PLGA, PCL or PLAPCL;
n and k are independently integers from 1 to 150;
p is an integer from 1 to 100;
W is a carbon atom, a C₁-C₆-alkyl group or an ether group comprising 1 to 6 carbon atoms;
z is an integer from 3 to 8;
wherein mol % of components a) to c) are expressed relative to the total number of moles of compounds a), b) and c).

15. Pharmaceutical kit comprising:
i) at least one hydrophilic degradable microsphere in association with a pharmaceutically acceptable carrier for administration by injection;
ii) an effective amount of buprenorphine or a pharmaceutically acceptable salt thereof; and
iii) optionally an injection device,
the hydrophilic degradable microsphere and the buprenorphine or a pharmaceutically acceptable salt thereof being packed separately, and wherein the hydrophilic degradable microsphere comprises a crosslinked matrix, the crosslinked matrix being based on at least the following components:
d) from 10 mol % to 90 mol % of a hydrophilic monomer of general formula (I):
(CH₂═CR₁)—CO-D (I)
wherein:
D is O—Z or NH—Z, with Z being —(CR₂R₃)ₘ—CH₃, —(CH₂—CH₂—O)ₘ—H, (CH₂—CH₂—O)ₘ—CH₃, —(CR₂R₃)ₘ—OH or —(CH₂)ₘ—NR₅R₆ with m being an integer from 1 to 30;
R₁, R₂, R₃, R₄, R₅ and R₆ are, independently of one another, hydrogen atom or a (C₁-C₆)alkyl group;
e) from 0.1 mol % to 30 mol % of a cyclic monomer of formula (II): 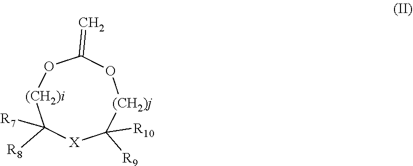
wherein:
R₇, R₈, R₉ and R₁₀ are, independently of one another, a hydrogen atom, a (C₁-C₆)alkyl group or an aryl group;
i and j are independently of one another an integer chosen between 0 and 2; and
X is a single bond or an oxygen atom;
and
f) from 5 mol % to 90 mol % of a linear or star-shaped degradable block copolymer cross-linker having a partition coefficient P of between −3 and 11.20, or a hydrophobic/hydrophilic balance R between 1 and 20, said degradable block copolymer cross-linker having the formula:
(CH₂═CR₁₁)—CO—Xₙ-PEGₙ-Xₖ—CO—(CR₁₁═CH₂) (IIIa); or
W(PEGₚ-Xₙ—O—CO—(CR₁₁═CH₂))_{z} (IIIc);
wherein
R₁₁ is independently a hydrogen atom or a (C₁-C₆)alkyl group;
X is independently PLA, PGA, PLGA, PCL or PLAPCL;
n and k are independently integers from 1 to 150;
p is an integer from 1 to 100;
W is a carbon atom, a C₁-C₆-alkyl group or an ether group comprising 1 to 6 carbon atoms;
z is an integer from 3 to 8;
wherein mol % of components a) to c) are expressed relative to the total number of moles of compounds a), b) and c).

16. The composition of claim 1, wherein the degradable block copolymer cross-linker c) is of general formula (Ma), wherein:
X=PLA, n+k=12 and p=13; or
X=PLAPCL, n+k=10 and p=13; or
X=PLAPCL, n+k=9 and p=13; or
X=PLAPCL, n+k=8 and p=13; or
X=PCL; n+k=8 and p=13; or
X=PLGA; n+k=12 and p=13; or
X=PCL, n+k=10 and p=4; or
X=PCL, n+k=12 and p=2.

17. The composition of claim 1, wherein the degradable block copolymer cross-linker c) is of general formula (IIIa) or (IIIc) wherein X represents PCL.
